SEQUENCE LISTING (42 sequences)

SEQ ID NO: 1 - DNA, 15 bp, Artificial Sequence
  chemically synthesized
ctgctggttttccac

SEQ ID NO: 2 - DNA, 14 bp, Artificial Sequence
  chemically synthesized
gttgaggggcatcg

SEQ ID NO: 3 - DNA, 17 bp, Artificial Sequence
  chemically synthesized
cctacgccaccagctcc

SEQ ID NO: 4 - DNA, 19 bp, Artificial Sequence
  chemically synthesized
gctgtatcgtcaaggcact

SEQ ID NO: 5 - DNA, 15 bp, Artificial Sequence
  chemically synthesized
gagaaagggctgcca

SEQ ID NO: 6 - DNA, 16 bp, Artificial Sequence
  chemically synthesized
gtagagatgcggtggt

SEQ ID NO: 7 - DNA, 12 bp, Artificial Sequence
  chemically synthesized
ctggtgttccat

SEQ ID NO: 8 - DNA, 15 bp, Artificial Sequence
  chemically synthesized
gaaggaggaaggagg

SEQ ID NO: 9 - DNA, 15 bp, Artificial Sequence
  chemically synthesized
ctccatggtgctcac

SEQ ID NO: 10 - DNA, 18 bp, Artificial Sequence
  chemically synthesized
acactagcatcgtatcag

SEQ ID NO: 11 - DNA, 15 bp, Artificial Sequence
  chemically synthesized
atcaatatttaacaa

SEQ ID NO: 12 - DNA, 19 bp, Artificial Sequence
  chemically synthesized
gcaaatactcaccatttgg

SEQ ID NO: 13 - DNA, 17 bp, Artificial Sequence
  chemically synthesized
gagtccttccacgatac

SEQ ID NO: 14 - DNA, 18 bp, Artificial Sequence
  chemically synthesized
caaaggtttggaatctgc

SEQ ID NO: 15 - DNA, 18 bp, Artificial Sequence
  chemically synthesized
ctcagcgtaagtgatgtc

SEQ ID NO: 16 - DNA, 18 bp, Artificial Sequence
  chemically synthesized
cagcatgaggaccatcag

SEQ ID NO: 17 - DNA, 18 bp, Artificial Sequence
  chemically synthesized
gtacacgtctctgtctgg

SEQ ID NO: 18 - DNA, 19 bp, Artificial Sequence
  chemically synthesized
caacatcagtctgataagc

SEQ ID NO: 19 - DNA, 20 bp, Artificial Sequence
  chemically synthesized
gaaacccagcagacaatgta

SEQ ID NO: 20 - DNA, 22 bp, Artificial Sequence
  chemically synthesized
agcctatggaattcagttctca

SEQ ID NO: 21 - DNA, 23 bp, Artificial Sequence
  chemically synthesized
tcagttttgcatagatttgcaca

SEQ ID NO: 22 - DNA, 22 bp, Artificial Sequence
  chemically synthesized
aactatacaacctactacctca

SEQ ID NO: 23 - DNA, 22 bp, Artificial Sequence
  chemically synthesized
cccctatcacgattagcattaa

SEQ ID NO: 24 - DNA, 23 bp, Artificial Sequence
  chemically synthesized
ctacctgcactataagcacttta

SEQ ID NO: 25 - DNA, 24 bp, Artificial Sequence
  chemically synthesized
actacctgcactgtaagcactttg

SEQ ID NO: 26 - DNA, 16 bp, Artificial Sequence
  chemically synthesized
cctgcactataagcac

SEQ ID NO: 27 - DNA, 18 bp, Artificial Sequence
  chemically synthesized
gtgaagctaacgttgagg

SEQ ID NO: 28 - DNA, 27 bp, Artificial Sequence
  chemically synthesized with added amino6t* reference label
gtcacctcagcgtaagtgatgtcggac

SEQ ID NO: 29 - DNA, 28 bp, Artificial Sequence
  chemically synthesized with added amino6t* reference label
gtcacctcagcgtaagtgatgtcgtgac

SEQ ID NO: 30 - DNA, 18 bp, Artificial Sequence
  chemically synthesized with added amino6t* reference label
ctcagcgtaagtgatgtc

SEQ ID NO: 31 - DNA, 17 bp, Artificial Sequence
  chemically synthesized with added amino6t* reference label
ctcagcgtaagtgatgc

SEQ ID NO: 32 - DNA, 27 bp, Artificial Sequence
  chemically synthesized with biotinT attached as a reference label
gtcacctcagcgtaagtgatgtcggac

SEQ ID NO: 33 - DNA, 28 bp, Artificial Sequence
  chemically synthesized with biotinT attached as a reference label
gtcacctcagcgtaagtgatgtcgtgac

SEQ ID NO: 34 - DNA, 28 bp, Artificial Sequence
  chemically synthesized
gtcacctcagcgtaagtgatgtcgtgac

SEQ ID NO: 35 - DNA, 22 bp, Artificial Sequence
  chemically synthesized
tggacatcacttacgctgagta

SEQ ID NO: 36 - DNA, 25 bp, Artificial Sequence
  chemically synthesized with added amino6t* reference label
gtcaccctgcactataagcacggac

SEQ ID NO: 37 - DNA, 23 bp, Artificial Sequence
  chemically synthesized
taaagtgcttatagtgcaggtag

SEQ ID NO: 38 - DNA, 23 bp, Artificial Sequence
  chemically synthesized
taaagtgcttatattgcaggtag

SEQ ID NO: 39 - DNA, 23 bp, Artificial Sequence
  chemically synthesized
taaagtgcttctagtgcaggtag

SEQ ID NO: 40 - DNA, 28 bp, Artificial Sequence
  chemically synthesized with amino6t* label
gtcaccaacatcagtctgataagcggac

SEQ ID NO: 41 - DNA, 29 bp, Artificial Sequence
  chemically synthesized with amino6t* label
gtcaccaacatcagtctgataagcgtgac

SEQ ID NO: 42 - DNA, 29 bp, Artificial Sequence
  chemically synthesized
gtcaccaacatcagtctgataagcgtgac

(19) United States
(12) Patent Application Publication
(10) Pub. No.: US 2009/0104614 A1
(43) Pub. Date: Apr. 23, 2009

(54) QUANTITATIVE MOLECULAR PROBES

(75) Inventors: Andrew Tsourkas, Philadelphia, PA (US); Antony Chen, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: THE TRUSTEES OF THE UNIVERSITY OF PENNSYLVANIA, Philadelphia, PA (US)

(21) Appl. No.: 12/282,612

(22) PCT Filed: Mar. 15, 2007
(86) PCT No.: PCT/US07/64099

§ 371 Date: Sep. 11, 2008

Related U.S. Application Data
(60) Provisional application No. 60/783,132, filed on Mar. 15, 2006.

(51) Int. Cl. C12Q 1/68; C07H 21/04; G01N 33/00

(52) U.S. Cl. 435/6; 536/24.31; 436/94

(57) ABSTRACT

In accordance with this invention, a molecular probe for detection of a nucleic acid target containing a preselected target sequence is constructed and has at least two sources of a signal: a conventional reporter source and a reference source in a form of a luminescent material, e.g., a fluorophore, quantum dot, fluorescent nanoparticle, or other fluorescent reference dye/nanoparticle/microparticle conjugated to the molecular probe.

### BACKGROUND OF THE INVENTION

1. Field of Invention

This invention relates to molecular probes for detecting and quantifying nucleic acid sequences.

2. Description of Related Art

Over the past several decades, numerous analysis tools have been developed to help identify the genetic variations that lead to the onset and progression of various diseases such as cancer. Ability to detect and quantify nucleic acids is invaluable in providing solutions to researchers and doctors.

Nucleic acids such as RNAs (e.g., microRNA, non-coding RNAs and mRNA) have been identified as potential targets for gene therapy, imaging, cell manipulation, diagnostics, etc. RNA is often the ideal target for imaging and therapeutic treatments because many disease states stem from the deregulation of RNA expression and/or defects in RNA splicing. For example, BRCA1 mRNA is often spliced incorrectly in breast cancer, a mutant SMN2 gene is often associated with spinal muscular atrophy, increased tau mRNA has been associated with Alzheimers disease, guanylyl cyclase C mRNA is considered a biomarker for colorectal cancer, and there are many other genes that have been definitively linked to a wide range of diseases. Recent advances have found that even non-coding RNAs can play an important role in chromatin organization, gene expression, and disease etiology and thus may serve as diagnostic markers.

MicroRNAs (miRNAs) are an abundant class of short non-coding RNAs, having about 22 nucleotides in length, that act as potent negative regulators of gene expression. MicroRNAs have been implicated in most major cellular processes including proliferation, apoptosis, developmental timing, haematopoiesis, and organogenesis. Similar to mRNA and non-coding RNA, aberrant miRNA expression has also been definitively linked to the pathogenesis of cancer and other diseases (e.g. neurological diseases, viral diseases, metabolic diseases, etc.), suggesting that miRNA could be an important target for a wide range of applications including drug development, diagnostics, and cell manipulation.

As noted earlier, a number of studies have recently connected the aberrant expression of miRNAs with the pathogenesis of cancer. Interestingly, over 52% of miRNA genes are in cancer-associated genomic regions or in fragile sites. In general, miRNA expression seems to be globally lower in tumors compared with normal tissue; however, there is also an extraordinary level of diversity across cancers, with large amounts of diagnostic information encoded within a relatively small number of miRNAs. An increasing number of studies show that the down-regulation of specific miRNAs is linked to different forms of cancer; examples include lung cancer (let-7), colorectal neoplasia (miR-143 and miR-145), chronic lymphocytic leukemia (miR-15 and miR-16), primary glioblastoma (miR-128 and -181) and breast cancer (miR-125). It should be noted that some reports have also shown a high-up-regulation of specific miRNAs in specific tumor types, including glioblastomas (miR-21, >100× control), lung cancer (cluster miR-17-92), thyroid carcinoma (miR-221, -222, and -146; 11-19× control), primary glioblastomas (miR-221; 2-5× control), breast cancer (miR-21), B-cell lymphomas (cluster miR-17-92; up to 82× control), and diffuse large B-cell lymphomas, (miR-155; 10-30× control). It is likely that the number of up-regulated miRNAs associated to specific tumors will only increase in the future as more miRNA profiles are examined and as new technologies for the high-resolution, high-specificity analysis of miRNA become available. Thus, the potential diagnostic value of miRNAs for cancer detection has been established.

Over the past several decades, numerous analysis tools have been developed to help identify the genetic variations that lead to the onset and progression of cancer. However, many commonly used approaches such as fluorescence in situ hybridization (FISH), real-time PCR, and microarrays do not provide complete spatial-temporal profiles of gene expression at the single cell level that are vital for understanding the role of genetic processing in cellular function. In order to conduct a more careful examination of the molecular basis of cancer, an imaging strategy must be employed that allows for the real-time visualization and quantification of endogenous mRNA in living cells.

Currently, the three most predominantly used methods for studying RNA expression levels are northern blots, RT-PCR and microarrays. However, these methods generally provide only the relative change in gene expression for a population of cells and not an absolute measure of RNA copies at the single cell level. Under many circumstances, it is the aberrant behavior of only a few cells or the stochasticity of RNA expression within a population that may be of interest. Arduous techniques such as single-cell RT-PCR can provide a closer look at RNA transcripts within single cells, but the RNA must still be extracted from the actual cell and processed prior to analysis. The shortcomings associated with RNA handling have been highlighted in several recent studies, which have shown that up to 90% of transcripts can be lost during RNA purification, cDNA synthesis, and other steps required for PCR.

Quantifying miRNA is particularly challenging because the short nature of miRNAs makes enrichment, ligation, and/or amplification steps technically challenging. Further, cross-hybridization is problematic for microarrays due to the sequence similarity between some miRNAs or large variations in G-C content of others (i.e., require very different hybridization temperatures). These shortcomings motivated the development of alternative approaches to detect miRNAs. For example, the RNA-primed, array based Klenow enzyme assay (RAKE)¹ and the miVana miRNA labeling kit (Ambion) only require enrichment, thus eliminating the need for miRNA amplification. Alternatively, the use of oligonucleotide capture probes demonstrates an improved specificity compared with glass-slide microarrays, presumably because hybridization occurred in solution as opposed to on a surface. However, these oligonucleotide-based probes still require PCR-amplification of the miRNA². A single-molecule method for the quantification of miRNA gene expression is available; but the technology is not widely accessible like flow cytometry¹. Although single cell microdissection techniques can potentially be combined with any of these approaches to provide single cell miRNA measures, large numbers of cells could not be analyzed in a high-throughput fashion.

In order to conduct a more careful examination of the molecular basis of cancer, an imaging strategy must be employed that allows for the real-time visualization and quantification of endogenous RNA (e.g. mRNA, non-coding RNA, and miRNA) in living cells.

Currently, there are two predominant methods for visualizing endogenous nucleic acids in living cells. The first approach utilizes two linear oligonucleotides, which are fluorescently labeled at their 5′ and 3′ end with donor and acceptor fluorophores, respectively, forming a FRET pair (two-probe FRET)^{3, 4}. Upon hybridization to adjacent sites on a target nucleic acid the two fluorophores are brought into close proximity and the resulting sensitized emission of the acceptor dye and quenching of the donor dye serve as an indicator for target detection. The second approach utilizes a single dual-labeled oligonucleotide probes with a fluorophore at one end and a quencher at the other also called molecular beacons⁵⁻¹⁰. U.S. Pat. No. 6,150,097 to Tyagi et al., and U.S. Pat. No. 5,925,517 to Tyagi et al. describe nucleic acid detection probes having a FRET and non-FRET fluorescence quenching and assays using such probes.

In the absence of complimentary targets, the oligonucleotide probe is designed to form a stem-loop structure, bringing the fluorophore and quencher into close proximity and resulting in a ‘dark’ non-fluorescent state. Upon hybridization, the fluorophore and quencher are forced apart and fluorescence is restored. Molecular beacons enable a homogenous assay format where background is low without the need to wash away free probes.

The significant enhancement in fluorescence achieved upon hybridization (i.e. large signal-to-background) has made molecular beacons the preferred method for nucleic acid detection in vitro and in vivo. Molecular beacons also have the additional benefit of possessing a higher specificity than linear oligonucleotides¹¹⁻¹³. An improved specificity will be particularly favorable when hybridizing to miRNA, due to the high number of miRNA with related sequences. Cross-hybridization has been identified as a problem for glass-slide microarray assays but seems to be less problematic for homogeneous assays.

Although molecular beacons obviously have several advantageous features for imaging RNA in living cells, they also possess several limitations. Specifically, conventional molecular beacons only provide a qualitative/relative measure of RNA expression and do not yield a rigorous quantification. Further, there is a high potential for false-negatives and false-positives. For example, a low fluorescent signal in a cell can indicate either low RNA expression or fewer molecular beacons in the cell. Similarly, a strong fluorescent signal can be interpreted as an up-regulation of the target RNA or a larger number of molecular beacons in the cell. Consequently, inhomogeneous transfections in each cell can make interpretation of fluorescence very difficult. This generally limits the use of molecular beacons to the study of RNAs that undergo dramatic changes in expression level. Although many of these problems can be avoided with microinjection only, then only a few cells can be studied at a time and therefore this approach does not scale well for high-throughout screening. Another problem faced by conventional molecular beacons is they immediately localize to the nucleus, where they emit a non-specific signal. This nuclear sequestration limits the number of probes in the cytoplasm available for hybridization. Also, the non-specific fluorescence that emanates from the nucleus makes image analysis and RNA quantification difficult.

Nonetheless, a number of studies have shown that molecular beacons can detect messenger RNA in living cells^{5, 7, 8, 10, 14-22}. In vivo applications have ranged from monitoring the distribution and transport of β-actin mRNAs in motile fibroblasts¹⁰, to imaging the expression level of multiple genes in single breast carcinoma cells simultaneously^{20, 22}, to the real-time monitoring of oncogene expression over extended lengths of time (48 hours)²². Although the results are extremely promising, numerous criteria must be considered when designing any molecular beacon. Perhaps the most significant limitation is that molecular beacon signals from individual cells are difficult to compare directly to each other because of the potential bias arising from the different number of beacons present across cells. Several research groups have tried to overcome some of these limitations by performing ratiometric analysis as the basis for fluorescence analysis, using an optically distinct molecular beacon or linear oligonucleotide as a reference probe^{7, 10, 20}. Normalization of fluorescence by ratiometric imaging not potentially allows for differentiating RNA expression levels from cell-to-cell, and for more accurate/precise monitoring of RNA expression trends.

As previously mentioned, however, current ratiometric imaging strategies require the administration of two fluorescent oligonucleotides, one with a nonsense sequence and one with the desired antisense sequence. A major drawback of using two unlinked reporters is the variance in intracellular lifetime that exists between hybridized and unhybridized oligonucleotides. Indeed, this variance in intracellular lifetime is the basis of generating contrast between cells expressing oncogenes from those not expressing oncogenes when using radionuclide-labeled oligonucleotides for PET/SPECT imaging of gene expression. In fluorescent-based studies, it is probable that different fluorophores could further influence intracellular lifetime depending on its charge, hydrophilicity, and size. A second drawback of the dual oligonucleotide approach is that varying ratios of each oligonucleotide in each cell, considered common in liposome/dendrimer-based transfection methods, can also cause errors in quantification of RNA. These shortcomings limit current ratiometric techniques to the use of microinjection as a means to introduce the probes into cells. Microinjection is invasive and only allows for the study of a small number of cells. A third drawback is that the two oligonucleotide probes used for ratiometric imaging do not necessarily co-localize making it difficult to perform true ratiometric imaging.

U.S. Patent Application Publication 20050019265A1 to Hammer et al. describes preparing polymersomes encapsulating emissive agents. However, this reference does not describe utilizing polymersomes for calibration or encapsulating molecular probes.

Therefore, there is still a need in the art to produce quantitative molecular probes for imaging RNA that are capable of being introduced into living cells with high efficiencies and fast kinetics.

All references cited herein are incorporated herein by reference in their entireties.

### BRIEF SUMMARY OF THE INVENTION

In a first aspect, the invention is a quantitative molecular probe for detection of a nucleic acid target containing a preselected target sequence, said quantitative molecular probe being capable of assuming a closed conformation and an open conformation, said quantitative molecular probe comprising:

a) a target complement sequence of from 7 to about 140 nucleotides complementary to said preselected nucleic acid target sequence having a 5′ terminus and a 3′ terminus;

b) optionally comprising an affinity pair comprising a first affinity moiety covalently linked to said 5′ terminus and a second affinity moiety covalently linked to said 3′ terminus, said affinity pair interacting sufficiently to hold said quantitative molecular probe in the closed conformation in the absence of said nucleic acid target;

c) a label pair comprising a first label moiety conjugated to at least one of a first nucleotide of said preselected nucleic acid target sequence or said first affinity moiety and a second label moiety conjugated to at least one of a second nucleotide of said preselected nucleic acid target sequence or said second affinity moiety, provided that said first nucleotide and said second nucleotide are separated by at least 2 nucleotides and wherein said label moieties interact to affect a measurable characteristic of at least one of said label moieties such that a difference in a reporter signal is produced when said quantitative molecular probe is in the closed conformation, wherein hybridization of said target complement sequence to said target sequence causes said quantitative molecular probe to assume its open conformation, in which said label moieties do not so interact; and

d) a reference label moiety conjugated to at least one of said target complement sequence, said first affinity moiety, said second affinity moiety, said first label moiety, or said second label moiety, wherein said reference label moiety produces a reference signal which is a detectable signal qualitatively distinct from the reporter signal produced by any one of said first label moiety, said second label moiety alone or in combination with each other in the closed conformation.

In accordance with this aspect of the invention, a molecular probe is constructed capable of quantitatively detecting nucleic acids in vitro, in fixed cells, in living cells, in tissue, in living subjects and other samples containing nucleic acids apparent to those skilled in the art. The molecular probe consists of an oligonucleotide-based probe, which is typically designed to form a stem-loop structure although a linear oligonucleotide can also be used. The oligonucleotide is labeled with a fluorophore ‘reporter’ at one end and a quencher or FRET acceptor at the other, analogous to a ‘molecular beacon’ (already commercially available); however, the oligonucleotide is also labeled with a second optically distinct ‘reference’ dye/nanoparticle/microparticle, which is selected such that it is unquenched regardless of the conformation of the probe.

The molecular probe has at least two sources of a signal (e.g., fluorescence): a conventional molecular beacon (or dual-labeled oligonucleotide) “reporter” source and “a reference” source in a form of a luminescent material, e.g., a fluorophore, quantum dot, fluorescent nanoparticle, or other fluorescent “reference” dye/nanoparticle/microparticle conjugated to the molecular beacon.

The attachment of a reference dye to a molecular beacon (or dual-labeled oligonucleotide) provides significant advantages as compared to current methods. These advantages include the following:

(1) the ability to monitor transfection efficiency since the reference dye is unquenched; this will reduce false-negatives by allowing for the differentiation between untransfected cells and low gene expression levels;

(2) improved quantification of targeted RNA by removing (e.g., through normalization) the impact of instrumental and experimental variability;

(3) improved quantification over time by ensuring the ratio of the reference dye to the reporter dye remains constant;

(4) improved spatial quantification within single cells by ensuring that the reference dye and the reporter dye co-localize;

(5) improved quantification from cell-to-cell by ensuring that an equal ratio of reference dye and reporter dye are present in each cell;

(6) the ability to remove (via normalization) the cell-to-cell variations in fluorescence that may result from non-uniform transfection efficiency; and

(7) improved signal-to-background in some embodiments (specifically a reduced background) due to quenching of the reporter dye by both the quencher molecule and the reference dye when the molecular beacon is in its stem-loop conformation.

Another unique advantage of this construct is the potential to attach targeting ligands (or other biomolecules, e.g., cell internalization ligands, e.g., Tat-peptide) and antisense molecular probes (e.g., beacons) to the surface of the reference dye. This offers additional options for delivering the probe into cells and may expand the use of the invention to in vivo imaging. In the case of in vivo imaging, it is envisioned that the reference dye (or label) (e.g., a nanoparticle or macromolecule embodiment) will allow for longer circulation times. In one approach, macroimaging may allow for localization of malignancies, after which the molecular beacon fluorescence can be imaged via intravital microscopy, endoscopy, or following a biopsy to quantify gene expression. If the reference dye consists of an MR nanoparticle (e.g., iron-oxide nanoparticle) labeled with fluorophores, then MRI can be used for probe localization prior to intravital microscopy

The unquenched reference dye/nanoparticle expands upon the versatility of the molecular beacon by not only improving the ability to accurately and sensitively detect RNA expression and localization (for the reasons described above), but it also provides a means for mRNA quantification. Specifically, the reference dye signal can be quantified to determine the number of probes in the cell and ratiometric imaging, comparing the emission of the ‘report’ dye to the ‘reference’ dye, provides a simple means to quantify the extent of probe hybridization to target RNA. These quantitative measurements can subsequently be used to calculate the exact copy number of RNA within single cells. An advantage of using a nanoparticle/macromolecule as the reference dye is it prevents nuclear localization. Thus, the construct is not sequestered in the nucleus and no non-specific signal is observed in the cell (e.g. nucleus), which is often the case with conventional molecular beacons.

The probe of the invention has significant advantages as a diagnostic tool over current technologies and will overcome the shortcomings of the conventional molecular beacons mentioned above. Specifically, in addition to the ‘reporter’ fluorophore and quencher, these new molecular probes will be conjugated to a second ‘reference’ fluorescent moiety (e.g., a fluorescent dye, a fluorescently labeled dendrimer or other macromolecule or protein, a quantum dot, a fluorescent silica nanoparticle, a fluorescent polystyrene nanoparticle, etc.), which will remain unquenched regardless of the conformation of the probe (i.e. hairpin, random coil or hybridized). The attachment of a reference dye to the probe will provide significant advantages in terms of the ability to make quantitative measurements of gene expression and in the ability to measure small changes in gene expression regardless of transfection/delivery efficiency. In a preferred embodiment, the quantitative molecular probe (QMP) is a quantitative molecular beacon (QMB) wherein all of the beneficial features of conventional molecular beacons for detecting specific nucleic acids within cells are maintained and a new feature to quantify the levels of beacons and targets within each cell is added.

In certain embodiments, the affinity pair is required and wherein said quantitative molecular probe is a quantitative molecular beacon and wherein said first label moiety is conjugated to said first affinity moiety and said second label moiety is conjugated to said second affinity moiety.

In certain embodiments, the affinity pair is not present and wherein said quantitative molecular probe is a quantitative linear oligonucleotide and wherein said first label moiety is conjugated to said first nucleotide of said preselected nucleic acid and said second label moiety is conjugated to said second nucleotide of said preselected nucleic acid target sequence.

In certain embodiments, the reference label moiety is a fluorophore selected from at least one of a nanoparticle, a microparticle, a quantum dot, a fluorescently labeled dendrimer, a fluorescent moiety or a fluorescently labeled moiety.

In certain embodiments, the first label moiety is a reporter fluorophore and said second label moiety is a quencher selected to substantially quench fluorescence of the reporter fluorophore in a closed conformation and said reference label moiety is a reference fluorophore, wherein said reference label moiety produces a signal at a wavelength sufficiently distinct from a first label moiety wavelength.

In certain embodiments, the reference label moiety is at least one of Cy3.5, Cy5, Cy5.5, ALEXA 660, ALEXA 680.

In certain embodiments, the target complement sequence is from 15 to 30 nucleotides.

In certain embodiments, the affinity pair comprises complementary oligonucleotide arm sequences 3 to 25 nucleotides in length.

In certain embodiments, the first label moiety is covalently linked to said first affinity moiety and said second label moiety is covalently linked to said second affinity moiety.

In certain embodiments, the reference label moiety is conjugated via covalent bonding or affinity bonding.

In certain embodiments, the label pair comprises either a FRET pair or a non-FRET pair.

In certain embodiments, the affinity pair comprises an antibody and an antigen.

In certain embodiments, the quantitative molecular probe is tethered to a solid surface.

In certain embodiments, the quantitative molecular probe is a unimolecular quantitative molecular probe.

In certain embodiments, the quantitative molecular probe is a bimolecular quantitative molecular probe consisting of a first molecule containing approximately half of said target complement sequence including said 5′ terminus, the first affinity moiety and the first label moiety; and a second molecule containing approximately half of said target complement sequence including said 3′ terminus, the second affinity moiety and the second label moiety.

In certain embodiments, the reference label moiety further comprises a targeting ligand.

In certain embodiments, the quantitative molecular probe is encapsulated in a polymersome vesicle comprising a shell comprising an amphiphilic polymer or in a liposome vesicle.

In one variant, the invention is an improvement of a molecular probe for detection of a nucleic acid target containing a preselected target sequence having:

a) a target complement sequence of from 7 to about 140 nucleotides complementary to said preselected nucleic acid target sequence, having a 5′ terminus and a 3′ terminus;

b) an affinity pair comprising a first affinity moiety covalently linked to said 5′ terminus and a second affinity moiety covalently linked to said 3′ terminus, said affinity pair interacting sufficiently to hold said molecular probe in the closed conformation in the absence of said nucleic acid target; and

c) a label pair comprising a first label moiety conjugated to said first affinity moiety and a second label moiety conjugated to said second affinity moiety, wherein said label moieties interact to affect a measurable characteristic of at least one of said label moieties such that a difference in a reporter signal is produced when said molecular probe is in the closed conformation, wherein hybridization of said target complement sequence to said target sequence causes said molecular probe to assume its open conformation, in which said label moieties do not so interact; wherein the improvement comprises:

having a reference label moiety conjugated to at least one of said target complement sequence, said first affinity moiety, said second affinity moiety, said first label moiety, or said second label moiety, wherein said reference label moiety produces a reference signal which is a detectable signal qualitatively distinct from the reporter signal produced by any one of said first label moiety, said second label moiety alone or in combination with each other in the closed conformation so that said molecular probe is adapted to quantitative detect a nucleic acid target.

In a second aspect, the invention is a method for a quantitative detection of a nucleic acid target, the method comprising:

providing the quantitative molecular probe of the invention and a nucleic acid target;

contacting said quantitative molecular probe with said nucleic acid target;

hybridizing said quantitative molecular probe to said preselected nucleic acid target sequence; and

detecting a reporter signal from at least one of said first label moiety and second label moiety and a reference signal from said reference label moiety to obtain a fluorescence ratio,

analyzing said fluorescence ratio and said reference fluorescence signal and thereby quantitatively detecting said nucleic acid target.

In a third aspect, the invention is a method for a quantitative determination of an effect of a substance on a nucleic acid target, the method comprising:

providing the quantitative molecular probe of the invention and a nucleic acid target;

contacting said quantitative molecular probe with said nucleic acid target;

hybridizing said quantitative molecular probe to said preselected nucleic acid target sequence to form a complex;

optionally contacting said nucleic acid target with said substance;

contacting a complex with said substance; and

detecting a reporter signal from at least one of said first label moiety and second label moiety and a reference signal from said reference label moiety to obtain a fluorescence ratio; and thereby quantitatively determining the effect of said substance on said nucleic acid target.

In a fourth aspect, the invention is calibration kit for detection of a signal from a quantitative molecular probe, said calibration kit comprising:

a plurality of encapsulated quantitative molecular probes comprising a quantitative molecular probes of the invention, wherein said quantitative molecular probe is encapsulated in at least one of a polymersome vesicle comprising a shell comprising an amphiphilic polymer or a liposome vesicle, wherein said encapsulated quantitative molecular probes have a predetermined amount of said quantitative molecular probes hybridized to a preselected nucleic acid target sequence.

In this invention, a method to routinely screen for the specific deregulation of RNA expression that occurs only in a small number of cells is provided.

### BRIEF DESCRIPTION OF SEVERAL VIEWS OF THE DRAWINGS

The invention will be described in conjunction with the following drawings in which like reference numerals designate like elements and wherein:

FIGS. 1A and 1B is a schematic representation of a preferred quantitative molecular probe-unimolecular probe having a reference label moiety and a label pair according to the invention, wherein the label pair provides a detectable signal based upon interaction of label moieties of the label pair in the “closed” (FIG. 1A) and “open” (FIG. 1B) conformation.

FIG. 2 is a schematic representation of a preferred quantitative molecular probe (QMP) of the invention having a fluorescent nanoparticle as a reference dye that is attached to the preferred hairpin-forming unimolecular probe (i.e., a molecular beacon). The unimolecular probe is shown in the “closed” conformation.

FIG. 3 is a schematic representation of a preferred QMP having a fluorescent nanoparticle as a reference dye that is attached to the preferred hairpin-forming unimolecular probe (i.e., a molecular beacon). The unimolecular probe is shown hybridized to a target nucleic acid and in the “open” conformation.

FIG. 4 illustrates a representative emission spectrum of a QMP composed of a quantum dot (max emission is 800 nm; QD800) as the reference dye and a “molecular beacon” labeled with Cal610 (max emission is 610 nm) and IBRQ quencher as the reporter probe. The emission spectrum is shown for QMPs in the presence and absence of complementary nucleic acid targets.

FIGS. 5A-5F are fluorescent images and ratiometric analysis of MCF-7 breast cancer cells microinjected with antisense c-myc QMPs (representative data). MCF-7 cells were either injected with antisense c-myc QMPs alone or in the presence of excess 2′-O-methyl antisense c-myc oligonucleotides targeting the identical sequence. Background subtracted fluorescent images of QD800 (FIG. 5A and FIG. 5D) were similar for all cells studied; however, while antisense c-myc QMPs injected alone elicited a detectable signal in the Cal610 channel (FIG. 5E), no signal was detected when 2′-O-methyl antisense were used to inhibit QMB hybridization (FIG. 5B). This is also evident in Cal610/QD800 ratiometric images (FIG. 5F and FIG. 5C respectively).

FIGS. 6A-6B are QMP microscope standardization curves. Antisense luciferase QMPs were microinjected into paraffin oil and the resulting bubble was imaged by fluorescence microscopy. FIG. 6A demonstrated that a linear correlation exists between total QD800 fluorescence and the number of quantum dots within each water-in-oil bubble. FIG. 6B demonstrated that a linear correlation also exists between the fluorescent ratio, Cal610:QD800, in each bubble and the number of target molecules per QMP in each bubble. This linear relationship holds until all of the molecular beacons are hybridized at which point the fluorescent ratio plateaus. The relationship between the fluorescent ratio and number of target molecules per QMP is independent of QMP concentration.

### DETAILED DESCRIPTION OF THE INVENTION

The invention is based on the discovery that by conjugating an additional label source to an oligonucleotide probe to serve as a reference, quantitative information on gene expression with spatial and temporal resolution can now be obtained.

Accordingly, the invention includes a quantitative molecular probe (QMP) for detection of a nucleic acid target containing a preselected target sequence, said quantitative molecular probe being capable of assuming a closed conformation and an open conformation and comprising:
In a first aspect, the invention is a quantitative molecular probe for detection of a nucleic acid target containing a preselected target sequence, said quantitative molecular probe being capable of assuming a closed conformation and an open conformation, said quantitative molecular probe comprising:

a) a target complement sequence of from 10 to about 140 nucleotides complementary to said preselected nucleic acid target sequence having a 5′ terminus and a 3′ terminus;

b) optionally comprising an affinity pair comprising a first affinity moiety covalently linked to said 5′ terminus and a second affinity moiety covalently linked to said 3′ terminus, said affinity pair interacting sufficiently to hold said quantitative molecular probe in the closed conformation in the absence of said nucleic acid target;

c) a label pair comprising a first label moiety conjugated to at least one of a first nucleotide of said preselected nucleic acid target sequence or said first affinity moiety and a second label moiety conjugated to at least one of a second nucleotide of said preselected nucleic acid target sequence or said second affinity moiety, provided that said first nucleotide and said second nucleotide are separated by at least 2 nucleotides and wherein said label moieties interact to affect a measurable characteristic of at least one of said label moieties such that a difference in a reporter signal is produced when said quantitative molecular probe is in the closed conformation, wherein hybridization of said target complement sequence to said target sequence causes said quantitative molecular probe to assume its open conformation, in which said label moieties do not so interact or interact at a lesser extent; and

d) a reference label moiety conjugated to at least one of said target complement sequence, said first affinity moiety, said second affinity moiety, said first label moiety, or said second label moiety, wherein said reference label moiety produces a reference signal which is a detectable signal qualitatively distinct from the reporter signal produced by any one of said first label moiety, said second label moiety alone or in combination with each other in the closed conformation.

Hybridization probes of the invention can be made from DNA, RNA, or some combination of the two. The probes may also include modified nucleotides. Modified internucleotide linkages are useful in probes comprising deoxyribonucleotides and ribonucleotides to alter, for example, hybridization strength and resistance to non-specific degradation and nucleases. The links between nucleotides in the probes may include bonds other than phosphodiester bonds, for example, peptide bonds. Modified internucleotide linkages are well known in the art and include methylphosphonates, phosphorothioates, phosphorodithionates, phosphoroamidites and phosphate ester linkages. Dephospho-linkages are also known, as bridges, between nucleotides and include siloxane, carbonate, carboxymethyl ester, acetamidate, carbamate, and thioether bridges. “Plastic DNA,” having for example N-vinyl, methacryloxyethyl, methacrylamide or ethyleneimine internucleotide linkages can also be used in probes (see e.g. Uhlmann and Peyman (1990) pp. 545-569) “Peptide Nucleic Acid” (PNA) is particularly useful because of its resistance to degradation by nucleases and because it forms a stronger hybrid with natural nucleic acids. (Orum et al. (1993); Egholm, et al. (1993) herein incorporated by reference).

The target complement sequence of the probe of the invention comprises from 8 to about 140 nucleotides complementary to said preselected nucleic acid target sequence and preferable from 12 to 30 nucleotides. Non-limiting examples of the target complement sequence are listed below:

| | |
|---|---|
| SEQ ID NO:1 | CTG CTG GTT TTC CAC (c-myc) |
| SEQ ID NO:2 | GTT GAG GGG CAT CG (c-myc) |
| SEQ ID NO:3 | CCTACGCCACCAGCTCC (k-ras) |
| SEQ ID NO:4 | GCTGTATCGTCAAGGCACT (k-ras) |
| SEQ ID NO:5 | GAGAAAGGGCTGCCA (Survivin) |
| SEQ ID NO:6 | GTAGAGATGCGGTGGT (Survivin) |
| SEQ ID NO:7 | CTGGTGTTCCAT (CCND1) |
| SEQ ID NO:8 | GAAGGAGGAAGGAGG (c-Src) |
| SEQ ID NO:9 | CTCCATGGTGCTCAC (Her/neu) |
| SEQ ID NO:10 | ACACTAGCATCGTATCAG (Her/neu) |
| SEQ ID NO:11 | ATC AAT ATT TAA CAA (ALF) |
| SEQ ID NO:12 | GCA AAT ACT CAC CAT TTG G (MyoD) |
| SEQ ID NO:13 | GAGTCCTTCCACGATAC (GAPDH) |
| SEQ ID NO:14 | CAA AGG TTT GGA ATC TGC (mmp-9) |
| SEQ ID NO:15 | CTCAGCGTAAGTGATGTC (Luc) |
| SEQ ID NO:16 | CAGCATGAGGACCATCAG (mam) |
| SEQ ID NO:17 | GTACACGTCTCTGTCTGG (TFF1) |
| SEQ ID NO:18 | CAACATCAGTCTGATAAGC (MiR-21) |
| SEQ ID NO:19 | GAAACCCAGCAGACAATGTA (MiR-221) |
| SEQ ID NO:20 | AGCCTATGGAATTCAGTTCTCA (MiR-146b) |
| SEQ ID NO:21 | TCAGTTTTGCATAGATTTGCACA (MiR-19a) |
| SEQ ID NO:22 | AACTATACAACCTACTACCTCA (let-7a) |
| SEQ ID NO:23 | CCCCTATCACGATTAGCATTAA (MiR-155) |
| SEQ ID NO:24 | CTACCTGCACTATAAGCACTTTA (MiR-20) |
| SEQ ID NO:25 | ACTACCTGCACTGTAAGCACTTTG (MiR-17-5p) |
| SEQ ID NO:26 | CCTGCACTATAAGCAC (MiR-20) |
| SEQ ID NO:27 | GTGAAGCTAACGTTGAGG (c-myc) |

In certain embodiments, probes described by Tyagi et al. in U.S. Pat. No. 5,925,517 and U.S. Pat. No. 6,150,097 (incorporated herein in their entireties) were modified to obtain the quantitative molecular probes of the invention.

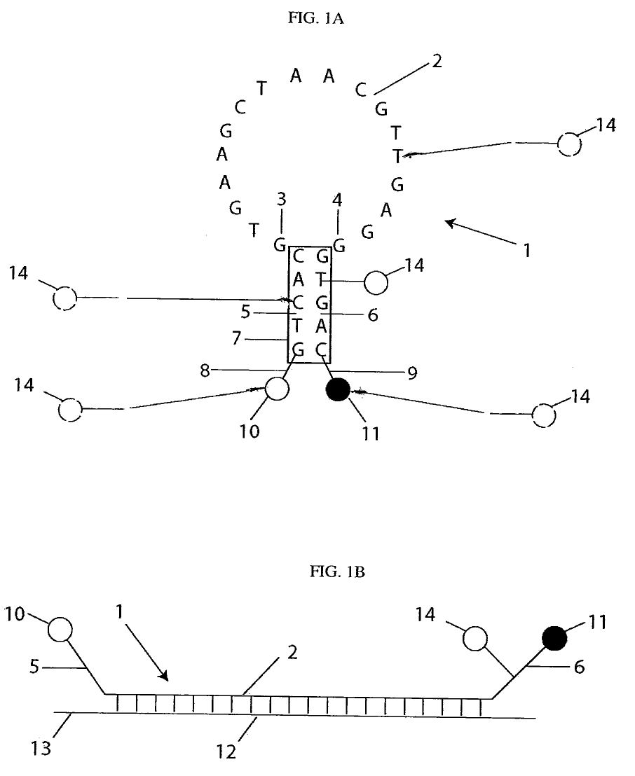

FIG. 1A schematically shows a unimolecular version of a quantitative molecular probe 1 with an interactive label pair and a reference label. Probe 1 includes a single-stranded target complement sequence 2 having a 5′ terminus and a 3′ terminus (3 and 4). Sequence 2 is complementary to a preselected target sequence contained within a nucleic acid target strand. Probe 1 can be considered as two strands, the bimolecular version, in which a single target complement sequence 2 is severed at about its midpoint. The following description describes probe 1 as so considered, that is, as the unimolecular version, for convenience. The description thus applies to both the bimolecular and unimolecular versions.

Extending from sequence 2, and linked thereto, are an affinity pair, herein depicted as oligonucleotide arms 5, 6. An affinity pair is a pair of moieties which have affinity for each other. Although a complementary nucleic acid sequences are preferred, as shown in FIG. 1A, other affinity pairs can be used. Examples include protein-ligand, antibody-antigen, protein subunits, and nucleic acid binding proteins-binding sites. Additional examples will be apparent to those skilled in the art. In some cases, use of more than one affinity pair may be appropriate to provide the proper strength to the interaction. The affinity pair reversibly interacts sufficiently strongly to maintain the probe in the closed state under detection conditions in the absence of target sequence but sufficiently weakly that the hybridization of the target complement sequence and its target sequence is thermodynamically favored over the interaction of the affinity pair. This balance allows the probe to undergo a conformational change from the closed state to the open state. Additionally, the affinity pair should separate only when probe binds to target and not when probe is non-specifically bound.

The mechanism by which the probe shifts from a closed to an open conformation will be described for the embodiment in which the affinity pair is complementary oligonucleotide arms, but the generalization to other affinity pairs will be apparent. Referring to FIG. 1A, arms 5, 6 are chosen so that under preselected assay conditions, including a detection temperature, they hybridize to each other, forming stem duplex 7, which is sometimes refer to as an arm stem. In the absence of target, association of arms 5, 6 is thermodynamically favored and maintains stem duplex 7, holding the probe 1 in the closed conformation depicted in FIG. 1A. In FIG. 1B, target complement sequence 2 is hybridized to target sequence 12 of target nucleic acid 13. Hybridization forms a relatively rigid double-helix of appropriate length. For the bimolecular version of the probe with interactive labels, it is a nicked helix. For probes of this invention formation of a helix by interaction of the target complement sequence and the target sequence is thermodynamically favored under assay conditions at the detection temperature and drives the separation of arms 5, 6, resulting in dissolution of stem duplex 7 and the maintenance of the open conformation depicted in FIG. 1B. Arm regions 5 and 6 do not interact with each other to form the stem duplex when target complement sequence 2 is hybridized to the target sequence 12. Because the interaction of the target complement sequence 2 with the target sequence 12 drives the separation of the arms 5 and 6, we sometimes refer to this mechanism as a “spring.” For certain embodiments of interactively labeled probes that are not allele-discriminating probes, the shift from the closed conformation to the open conformation occurs when the target complement sequence hybridizes to the target sequence despite the presence of a nick or the presence of one or more nucleotide mismatches. Importantly, non-specific binding of the probe does not overcome the association of the arms in this manner. This feature leads to very low background signal from inappropriately “opened” probes.

The affinity pair illustrated in the preferred embodiment of FIGS. 1A and 1B is a pair of complementary nucleic acid sequences. Arms 5, 6 are chosen so that stem duplex 7 (FIG. 1A) is a smaller hybrid than the hybrid of target complement sequence 2 and target sequence 12 (FIG. 1B). In the bimolecular version, stem duplex 7 should be smaller than either portion of the nicked helix, each of which is approximately half the length of 2. If that limitation is satisfied, each half of 2 contains “approximately half” of target complement sequence 2. Other affinity pairs, as indicated, may be conjugated to the target complement sequence, in some cases through non-complementary arms or to non-nucleic acid arms. Appropriate affinity pairs may be conjugated to the target complement sequence by methods known in the art. Preferably, the affinity pair is covalently linked directly to the target complement sequence.

A probe having interactive labels as described above has a measurable characteristic, for example, a signal, due to the label pair. Probe 1 includes label moieties 10, 11 conjugated to and forming part of probe 1 at the 5′ and 3′ termini, respectively, of the stem duplex 7. Label moieties 10, 11 are placed such that their proximity, and therefore their interaction with each other, is altered by the interaction of arms 5, 6. Label moieties 10, 11 could be conjugated elsewhere to arms 5, 6 or to sequence 2 near its linkage with the stem 7, that is, close to arms 5, 6. Some label moieties will interact to a detectably higher degree when conjugated internally along the arms. This is because they will not be affected by unraveling of the termini.

In certain embodiments, probe 1 can consist of the target complimentary sequence with no affinity labels (e.g. 5 and 6), i.e. a linear version of probe 1. In this embodiment, label moieties 10, 11 are conjugated to sequence 2 near or at the 5′ and 3′ terminus. In this embodiment, the “closed” conformation of the probe refers the Probe 1 when it assumes a random coil conformation.

For all embodiments of the probe, more than one pair of label moieties may be used. Further, there is no requirement for a one-to-one molecular correspondence between members of a label pair, especially where one member can affect, or be affected by, more than one molecule of the other member. Label moieties suitable for use in probes of this invention interact so that at least one moiety can alter at least one physically measurable characteristic of another label moiety in a proximity-dependent manner. The characteristic signal of the label pair is detectably different depending on whether the probe is in the open conformation or the closed conformation.

For example, referring to FIGS. 1A and 1B, the preferred label moieties are a FRET pair or a fluorophore-quencher pair, most preferably fluorophore 10 and quencher 11. In that embodiment, the characteristic signal is fluorescence of a particular wavelength. When probe 1 is in the closed state (FIG. 1A), label moiety 11 quenches fluorescence from moiety 10. When moiety 10 is stimulated by an appropriate frequency of light, a fluorescent signal is generated from the probe at a first level, which may be zero. Probe 1 is “off.” When probe 1 is in the open state (FIG. 1B), label moiety 10 is sufficiently separated from label moiety 11 that fluorescence resonance energy transfer between them is substantially, if not completely, precluded. Label moiety 11 is therefore unable to quench effectively the fluorescence from label moiety 10. If moiety 10 is stimulated, a fluorescent signal of a second level, higher than the first is generated. Probe 1 is “on.” The difference between the two levels of fluorescence is detectable and measurable. Utilizing fluorescent and quencher moieties in this manner, the probe is only “on” in the “open” conformation and indicates that the probe is bound to the target by emanating an easily detectable signal. The conformational state of the probe alters the signal generated from the probe by regulating the interaction between the label moieties.

In embodiments wherein the affinity pair is complementary oligonucleotide arms, lengths of target complement sequences and arm sequences are chosen for the proper thermodynamic functioning of the probe under the conditions of the projected hybridization assay. Persons skilled in hybridization assays will understand that pertinent conditions include probe, target and solute concentrations, detection temperature, the presence of denaturants and volume excluders, and other hybridization-influencing factors. The length of a target complement sequence can range from 7 to about 140 nucleotides, preferably from 10 nucleotides to about 30 nucleotides. If the probe is also an allele-discriminating probe, the length is more restricted, as is discussed later. For bimolecular embodiments, each portion of the target complement sequence should have a length of at least 10 nucleotides. The lower limit is set by the minimum distance at which there is no detectable difference in the measurable characteristic (or characteristic signal) affected by the interaction between the label moieties used when the probe is closed, from when the probe is opened. Thus, the minimum length of the target complement sequence 2 for a particular probe depends upon the identity of the label pair and its conjugation to the probe. In certain embodiments, the label moiety is the fluorescent moiety 5->(2-aminoethyl)amino-naphthalene-1-sulfonic acid (EDANS) and the quenching moiety is 4-(4-dimethylaminophenylazo)benzoic acid (DABCYL). For EDANS and DABCYL, quenching is essentially eliminated by a separation of 60 Angstroms, which is equivalent in length to about 20 nucleotide pairs in a double-helical nucleic acid.

The conjugation of the label moieties to any location on the probe must be stable under the conditions of the assay. Conjugation may be covalent, which is preferred. Examples of non-covalent conjugation include, without limitation, ionic bonding, intercalation, protein-ligand binding and hydrophobic and hydrophilic interactions. Appropriately stable means of association of label moieties to the probes will be apparent to those skilled in the art. The use of the term “conjugation” herein encompasses all means of association of the label moieties to the probe which are stable under the conditions of use. We consider stably conjugated label moieties to be included within the probe molecule to which they are conjugated.

In certain embodiments, label moieties are conjugated to the probes by covalent linkage through spacers, preferably linear alkyl spacers. The nature of the spacer is not critical. For example, EDANS and DABCYL may be linked via six-carbon-long alkyl spacers well known and commonly used in the art. The alkyl spacers give the label moieties enough flexibility to interact with each other for efficient fluorescence resonance energy transfer, and consequently, efficient quenching. The chemical constituents of suitable spacers will be appreciated by persons skilled in the art. The length of a carbon-chain spacer can vary considerably, at least from 1 to 15 carbons. However, in the case of multiple labels conjugated to an arm in a “bunch of grapes” configuration, a multiply bifurcated spacer is desirable.

For allele-discriminating probes that have non-interactive labels, labels may be conjugated to the probes as described above. However, radioactive labels may be incorporated in the probes by synthesis with radioactive nucleotides or by a kinase reaction, as is known in the art.

Luminescent label moieties to be paired with appropriate quenching moieties can be selected from any one of the following known categories: a fluorescent label, a radioluminescent label, a chemiluminescent label, a bioluminescent label and an electrochemiluminescent label.

The use of multiple quenching moieties with a single luminescent moiety will increase quenching. In this instance a label pair comprises one fluorescent moiety “paired” to several quenching moieties. Other useful label pairs include a reporter enzyme and appropriate inhibitor.

In certain embodiments, labels are chosen such that fluorescence resonance energy transfer is the mode of interaction between the two labels. In such cases, the measurable physical characteristics of the labels could be a decrease in the lifetime of the excited state of one label, a complete or partial quenching of the fluorescence of one label, an enhancement of the fluorescence of one label or a depolarization of the fluorescence of one label. The labels could be excited with a narrow wavelength band of radiation or a wide wavelength band of radiation. Similarly, the emitted radiation could be monitored in a narrow or a wide range of wavelengths, either with the aid of an instrument or by direct visual observation. Examples of such pairs are fluorescein/sulforhodamine 101, fluorescein/pyrenebutanoate, fluorescein/fluorescein, acridine/fluorescein, acridine/sulforhodamine 101, fluorescein/ethenoadenosine, fluorescein/eosin, fluorescein/erythrosin and anthranilamide-3-nitrotyrosine/fluorescein. Other such label pairs will be apparent to those skilled in the art.

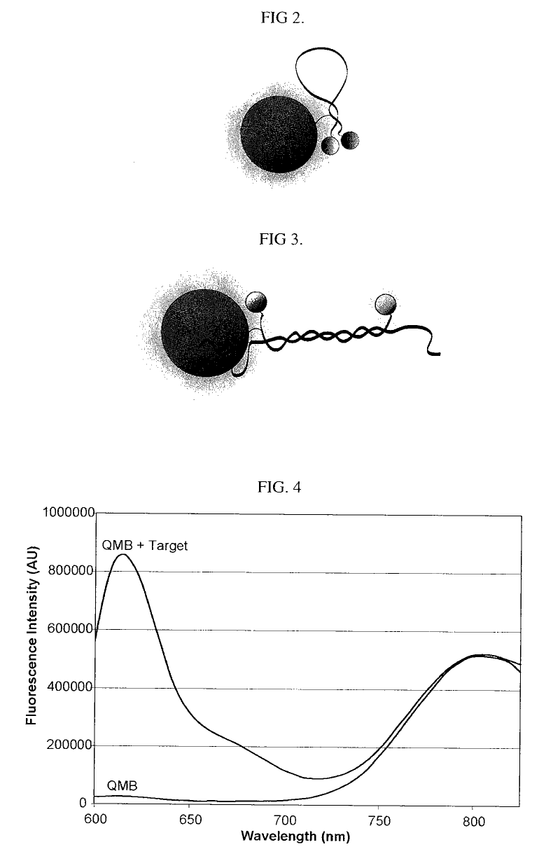

Further, the probe 1 includes a reference label moiety 14 conjugated to at least one of said target complement sequence, said first affinity moiety, said second affinity moiety, said first label moiety, or said second label moiety, wherein said reference label moiety produces a detectable signal qualitatively distinct from a signal produced by any one of said first label moiety, said second label moiety alone or in combination with each other in the closed conformation. A representative schematic of the probe of the invention, termed ‘quantitative molecular probe’ (QMP), in the closed conformation is shown in FIG. 2. A representative schematic of the probe of the invention in the open conformation is shown in FIG. 3.

As a precursor to the QMP, a molecular beacon or a linear oligonucleotide sequence having a label pair is modified or synthesized to possess a functional group (e.g., amine, thiol, biotin, etc.) or other means of attachment within its stem (7), loop (2), or linked to the label moieties (10 or 11) using chemistries known to those experienced in the art.

Next, a reference label moiety 14 is conjugated to the precursor to form the QMP of the invention. The reference label can consist of a fluorescent label, a radioluminescent label, a chemiluminescent label, a bioluminescent label or an electrochemiluminescent label. The reference label can be conjugated covalently or non-covalently to the precursor. FIG. 1A provides non-limiting examples of the placement of the reference label moiety 14, which is shown by a broken line. The reference label is chosen such that its signal can be detected regardless of the conformation of the probe. In preferred embodiments, the label moieties 10 and 11 on probe 1 will not interact or alter the signal of the reference label 14, although this is not limiting. In alternative embodiments, the label moieties 10 and 11 may interact or alter the signal of the reference label 14.

The probe of the invention can be prepared using standard techniques.

Molecular beacons with the desired functional group can be synthesized using standard oligonucleotide synthesis techniques or purchased (e.g., from Integrated DNA Technologies).

The reference dyes can be purchased (e.g., from Quantum dots; Qdot Corp. and Evidentech, fluorescent polystyrene particles; Invitrogen and Polysciences, etc.) or synthesized using materials that are readily available (e.g., dendrimers; Sigma, Fluorescent dyes; Molecular Probes and Amersham, etc.).

Cross-linking reagents are commercially available or can be easily synthesized. Targeting, cell internalization, or other ligands are commercially available or can be easily synthesized or produced using standard biological techniques.

Additional modifications include various available oligonucleotide chemistries and various fluorescent nanoparticles and dyes for the reference dye (e.g., quantum dots, fluorescently labeled dendrimers, fluorescently labeled iron-oxide nanoparticles, fluorescently labeled proteins, fluorescent proteins, fluorescent silica nanoparticles, fluorescent polystyrene nanoparticles, and any other fluorescent molecules, proteins, polymers, nanoparticles, microparticles, etc).

Also, QMPs labeled with targeting agents and/or cell internalization agents and/or other ligands (small molecules, peptides, proteins, etc.) are contemplated in this invention. Some cell internalization agents do not need to be physically attached to the QMP but have a similar function and should also be included as alternative embodiments. A person skilled in the art would be able to make such embodiments using methods known in the art without undue experimentation.

The performance of probes contemplated in this invention can be evaluated by determining (1) the ability to detect two signals, one from the reference moiety and one from the molecular beacon reporter moiety; (2) an change (enhancement of loss) in the reporter signal when the QMP is in the presence of complimentary target nucleic acids; (3) the ability to detect the reference signal regardless of whether the QMP is in the presence or absence of complimentary nucleic acid targets.

QMPs can be introduced into cells via microinjection or by using targeting, cell internalization, or by using ligands, polymers, liposomal agents, transfection agents, or other delivery agents known to those experienced in the art. These delivery agents can either be directly linked to the QMP or simply mixed with the QMP depending on the internalization strategy. If QMPs are not being used for intracellular imaging but for simply detecting RNA then no internalization strategy is needed. QMPs can be attached to a solid matrix using covalent, ionic, or hydrogen bonding. In certain embodiments, metal coordination bonding can also be used. Covalent bonding of QMPs is preferred. Solid matrixes can be polymeric, metallic, ceramic or a combination of the above.

Typical doses for cell studies generally range from 0.1 nM to 1 mM, but QMPs can of course be used outside this range. For animal studies, the dose would be similar to other agents using the same reference nanoparticle/dye (e.g. ˜1-10 mg/kg if fluorescently labeled iron-oxide nanoparticles are used).

In certain embodiments, the quantitative molecular probe is encapsulated in a polymersome vesicle comprising a shell comprising an amphiphilic polymer or in a liposome vesicle.

Encapsulated Quantitative Molecular Probes-Polymersomes QMP and Liposomes QMP

Polymersomes are vesicles made using amphiphilic diblock and multiblock copolymers where at least one block is hydrophobic and at least one block is hydrophilic. In particular, these diblock and multiblcok copolymers can form thick-walled vesicles when placed in an aqueous media. Polymersomes can be stably prepared by a number of techniques which are common to liposomes (Lee et al., Biotechnology and Bioengineering, vol. 73, no. 2, Apr. 20, 2001). Processes such as film rehydration, sonication, and extrusion can generate many-micron giant vesicles as well as monodisperse vesicles with diameters as small as 100 nanometers.

Liposomes are vesicles made of lipid bilayers, mostly phospholipids. Preparations of liposomes are well known in the art.

Polymersomes are constructed using block copolymers which are macromolecules that are comprised of two or more polymer blocks differing in composition that are generally covalently bonded. Diblock copolymers typically comprise two covalently bonded polymer blocks differing in composition. In amphiphilic block copolymers, the two blocks have very different interactions with water. Amphiphilic diblock copolymers generally have one block soluble in water, and the other block essentially water insoluble.

A variety of biodegradable and biocompatible polymers can potentially be utilized as polymer segments in polymersomes. These include the following where FDA approved biodegradable polymers are indicated with a “*”: *polyglycolides (PGA), *polylactides (LPLA and DPLA), *polycaprolactone, *polydioxanone (PDO of PDS), *poly(lactide-co-glycolide) (PGA-LPLA), *polyanhydrides, *polyorthoesters, *poly(amino acids) and “pseudo”-poly(amino acids), *polyhydroxybutyrate (PHB), *polyhydroxyvalerate (PHV); polycyanoacrylates, polyphosphazenes, polyphosphonates, polyiminocarbonates, polyamines, polyolefins, polystyrene, polyoxyethylene, thermoset amino proteins, polysaccharides, polymethylmethacrylate (PMMA), polytetrafluoroethylene (PTFE), polyurethane, polyvinylchloride (PVC), polydimethylsiloxane (PDMS), polyesters, nylons, lignin-based biodegradable polymers, biodegradable polymers from soybeans, soy protein-based plastics, loose fill from corn, polymers based on synthetic genes, and bacterially-produced polymers such as polyhydroxyalkanoates. Biodegradable polymersomes are discussed in several publications (Meng, et. al., Macromolecules 36, 2003; 3004. F. Najafi, M. N. Sarbolouki, Biomaterials 24, March, 2003; 1175-1182).

In some embodiments, the hydrophilic polymer block is characterized as a composition that has a positive free energy change of transfer from water to a non-polar solvent such as hexane, cyclohexane, pentane, or toluene, relative to the free energy change for transferring glycine from water to the same non-polar solvent (see Radzicka, A. & Wolfenden, R., Biochemistry 26, 1664 (1988)). Some hydrophilic polymers comprise ionically polymerizable polar units. Ionically polymerizable polymers may be derived from units of one or more alkyl oxide monomers. In certain embodiments, the alkyl oxide monomers can be ethylene oxide, propylene oxide, or combinations thereof. In one particularly preferred embodiments, the hydrophilic polymer block comprises poly(ethylene oxide). In yet other embodiments, the volume fraction of the hydrophilic polymers in the plurality of amphiphilic block copolymers is typically less than about 0.40.

In some embodiments, the hydrophilic polymer block is a polyalkylene glycol. In certain embodiments, the polyalkylene glycol suitable for the hydrophilic component in the block copolymer of the present invention is polyethylene glycol, monoalkoxy polyethylene glycol, monoacyloxy polyethylene glycol, or any combination thereof.

In certain preferred compositions, the number average molecular weight of the hydrophilic polymer block in the range of 200 to about 20,000 Daltons, and, in some embodiments, preferably in the range of about 1,000 to about 15,000 Daltons. For some compositions, the content of the hydrophilic component is within the range of about 40 to about 80 weight percent, and in some embodiments, preferably about 40 to about 70 weight percent, based on the total weight of the block copolymer. In certain embodiments the content of the hydrophilic component may be less than about 40 weight percent of the block copolymer. In some embodiments a hydrophilic homopolymer having a molecular weight about the same as the amphiphilic block can be added to the amphiphilic block copolymer to form the polymersomes. In other embodiments, the weight ratio of the hydrophilic homopolymer to the hydrophilic block can be in the range of from about 20:80 to about 80:20, as long as the overall hydrophilic content of the homopolymer and block is within the range of about 40 to about 80 weight percent, and in some embodiments, preferably about 40 to about 70 weight percent, based on the total weight of the block copolymer and homopolymer.

In certain embodiments, the hydrophobic polymer is characterized as being insoluble in water. In some embodiments, the hydrophobic polymer is characterized as a composition having a negative free energy change of transfer from water to a non-polar solvent such as hexane, cyclohexane, pentane, or toluene, relative to the free energy change for transferring glycine from water to the same non-polar solvent (see Radzicka, A. & Wolfenden, R., Biochemistry 26, 1664 (1988)). Some preferred hydrophobic polymers include polyethylethylene, poly(butadiene), poly(.beta.-benzyl-L-aspartate), poly(lactic acid), poly(propylene oxide), poly(.epsilon.-caprolactam), oligo-methacrylate, and polystyrene. In yet other embodiments, the hydrophobic polymer comprises polymerized units selected from ethylenically unsaturated monomers, such as poly(isoprene) (“PI”) and polyethylenepropylene (“PEP”).

In certain of these embodiments, the ethylenically unsaturated monomers are hydrocarbons. In certain preferred embodiments, the hydrophobic polymer comprises polyethylethylene or poly(butadiene).

In yet other embodiments, the hydrophobic polymer component may be a biodegradable block including polylactides, polycaprolactone, copolymers of lactide and glycolide, copolymers of lactide and caprolactone, copolymers of lactide and 1,4-dioxan-2-one, polyorthoesters, polyanhydrides, polyphosphazines, poly(amino acid)s or polycarbonates. In certain embodiments, the molecular weight of the hydrophobic polymer component is preferably within the range of about 500 to about 20,000 Daltons, and, in some preferred embodiments, from about 1,000 to about 10,000 Daltons.

Polymersomes are constructed using block copolymers which are macromolecules that are comprised of two or more polymer blocks differing in composition that are generally covalently bonded. In certain embodiments, the polymer may contain a fluorocarbon block that is characterized as being insoluble in water. In some embodiments, the fluorocarbon polymer is characterized as a composition having a negative free energy change of transfer from a fluorocarbon phase to water as well as a negative free energy change of transfer from a fluorocarbon phase to a non-polar solvent such as hexane, cyclohexane, pentane, or toluene, relative to the free energy change for transferring glycine from water to the same non-polar solvent (see Radzicka, A. & Wolfenden, R., Biochemistry 26, 1664 (1988)). Some preferred fkuorocarbon polymers include perfluoinated derivatives of polyethylethylene, poly(butadiene), poly(P-benzyl-L-aspartate), poly(lactic acid), poly(propylene oxide), poly(.epsilon.-caprolactam), oligo-methacrylate, and polystyrene. In yet other embodiments, the fluorocarbon polymer comprises polymerized units selected from extensively fluorinated unsaturated monomers, such as poly(fluoroisoprene) (“PI”) and poly(fluoroethylenepropylene) (“PEP”).

Some preferred polymersomes comprise poly(ethylene oxide)-polyethylethylene, poly(ethylene oxide)-poly(butadiene), or poly(ethylene oxide)-poly(lactic acid) block copolymers. Other polymersomes include block copolymers disclosed in U.S. Pat. No. 6,569,528 which comprising polyethylenimine as a hydrophilic block and aliphatic polyesters as a hydrophobic block and poly(oxyethylene)-poly(ox-ypropylene) block copolymers disclosed in U.S. Pat. No. 6,060,518.

As used herein, PEO is polyethylene oxide, PEE is polyethylethene, PB or PBD is polybutadiene. PEE is typically provided by catalytic hydrogenation of butadiene polymers that include more than about 50 percent of the butadiene repeat units in the 1,2 configuration. Catalytic hydrogenation of 1,2 polybutadiene is described by J H Rosedale et al., J. Am. Chem. Soc. 110, 3542 (1988), and U.S. Pat. No. 5,955,546.

The polymersomes of the instant invention typically self-assemble into unique structures in melts (pure polymer solutions) and in aqueous mixtures. While not wanting to be bound by any particular theory of operation, it is believed that thermodynamics (entropic effects arising from non-covalent forces—ionic, hydrogen bonding, Van der Waals interactions) drives the self assembly of the block copolymers into these unique structures; structures such as lamellar phases (alternating layers of polymer blocks) or aqueous spherical (or rod like or worm-like) micelles, in which all of the molecules are clustered in a sphere in a single layer, with the hydrophilic parts pointed outward and the hydrophobic domains pointed inward, vary, inter alia, with molecular weight, chain conformation, and chemistry of the polymer. The solubility of the block copolymers is characterized generally by the overall interaction parameter, which is a relative mixing parameter; higher values of .chi. typically lead to more immiscible systems, and a greater tendency of the polymers to strongly segregate into structured phases. The other parameter is the composition of the block copolymer—the volume fraction of each block in the vesicle membrane. The phase of matter formed by the polymers depends in a complex way upon total polymer molecular weight, block composition, polymer conformation, the mixing parameter, temperature, and solvent.

Preparation of Polymersomes

Polymersomes can be prepared and processed by a number of methods known to one skilled in the art. These processes are analogous to techniques commonly practiced in the preparation of liposomes and include film rehydration, sonication, extrusion, mechanical shaking, freeze drying, freeze thawing, micro-emulsification, solvent dispersion, pH-induced vesiculation, ion/enzyme/ligand induced fusion, water-in-organic phase, double emulsion, reverse-phase evaporation and detergent solubilization techniques (see R. R. C. New, Liposomes: A Practical Approach. D. Rickwood, B. D. Hames, Eds., The Practical Approach Series; Oxford University Press, Oxford, UK, 1997). Giant vesicles of OE7 and OB2 spontaneously bud off of either rehydrated films or bulk copolymer. Electroformation of OE7, in which thin films are formed on two parallel platinum wires by chloroform evaporation, requires an oscillating voltage of somewhat higher amplitude (10 V, 10 Hz) than typically used for phospholipids such as SOPC (3 V, 1-10 Hz) to drive the budding process. The necessity of a higher driving voltage in electroformation likely reveals a higher lamellar viscosity. This is also manifested in relatively slow dynamics of osmotically induced vesicle shape changes of the sort described by in Discher, B. M.; Won, Y. Y.; Ege, D. S.; Lee, J. C.; Bates, F. S.; Discher, D. E.; Hammer, D. A., Science 284, 1143-1146 (1999).

Solutions of block copolymers used for vesicle formation, depending on the process used, can range from pure water to 250 mM sucrose or physiological PBS. Although unilamellar vesicles predominate in electroformed preparations, multilamellar vesicles that exhibit an enhanced edge contrast also have a tendency to form in the various methods. The passage of vesicles through a filter with pores of 0.1 mm diameter can be used, with or without sonication and freeze-thaw, to generate a very narrow distribution of vesicle sizes with retained contents. Multi-generational polymersomes, i.e., smaller polymersomes within larger polymersomes, are also prepared by these methods.

Because of certain similarities in mechanical properties between polymersomes prepared by various methods, no further process distinction has been made in characterizations such as long-term stability. In formal studies as well as more casual observation, it has been observed that polymersomes in dilute suspension maintain their contents and a stable size distribution for a month or longer. In contrast, it has been found that SOPC vesicles suspended under the same conditions lose their phase contrast within a day: nominally, t.sub.½.about.10-20 hours. Because the stability of a copolymer structure such as the lamellar phase is well appreciated as depending on the product of the Flory interaction energy, .chi., and chain length, N, one would expect that the polymersomes are not only more stable mechanically but also that they have a much lower critical micellization concentration (CMC) than lipids, given the same parameter.

In certain embodiments, the block co-polymer assemblies of the instant invention can have cross-linking. Cross-linking can stabilize to polymersome structure. For example, Ahmed, et al, Langmuir 19, 6505 (2003) have studied amphiphilic diblocks comprising hydrophilic poly(ethylene oxide) and cross-linkable hydrophobic polybutadiene. Cross-linkable compositions can be blended with non-cross-linkable compositions in certain embodiments. Vesicles constructed of cross-linked bock copolymers can be dehydrated and rehydrated without compromising the polymersome structure. See Discher, et. al, J. Phys. Chem. B, 106, 2848 (2002). This feature is advantageous in producing, storing, and later using polymersome compositions in end uses such as imaging, diagnostics, and therapeutics.

Imaging Agents

The polymersome containing QMP of the invention may comprise an additional visible- or near infrared-emissive agent that is dispersed within the polymersome membrane. In certain embodiments, the emissive agent emits light in the 700-1100 nm spectral regime. In other embodiments, at least one emissive agent comprises a porphyrin moiety. Other emissive agents include a porphycene-, rubyrin-, rosarin-, hexaphyrin-, sapphyrin-, chlorophyll-, chlorin-, phthalocynine-, porphyrazine-, bacteriochlorophyll-, pheophytin-, or texaphyrin-macrocyclic-based component, or a metalated derivative thereof.

The emissive agent may be a laser dye, fluorophore, lumophore, or phosphor in certain embodiments. A laser dye according to the invention is any organic, inorganic, or coordination compound that has the ability to lase. Suitable laser dyes include those found in Birge, R R, Duarte, F J, Kodak Optical Products, Kodak Publication JJ-169B (Kodak Laboratory Chemicals, Rochester, N.Y. (1990). Representative laser dyes include p-terphenyl, sulforhodamine B, p-quaterphenyl, Rhodamine 101, curbostyryl 124, cresyl violet perchlorate, popop, DODC iodide, coumarin 120, sulforhodamine 101, coumarin 2, oxozine 4 perchlorate, coumarin 339, PCM, coumarin 1, oxazine 170 perchlorate, coumarin 138, nile blue A perchlorate, coumarin 106, oxatine 1 perchlorate, coumarin 102, pyridine 1, coumarin 314T, styryl 7, coumarin 338, HIDC iodide, coumarin 151, PTPC iodide, coumarin 4, cryptocyanine, coumarin 314, DOTC iodide, coumarin 30, HITC iodide, coumarin 500, HITC perchlorate, coumarin 307, PTTC iodide, coumarin 334, DTTC perchlorate, coumarin 7, IR-144, coumarin 343, HDITC perchlorate, coumarin 337, IR-NO, coumarin 6, IR-132, coumarin 152, IR-125, coumarin 153, boron-dipyrromethere, HPTS, flourescein, rhodamine 110, 2,7-dichlorofluorescein, rhodamine 65, and rhodamin 19 perchlorate, rhodamine b, where the laser dye is modified by addition of a hydrophobic substitutent, and the laser dye being substantially dispersed within the polymersome membrane.

In some embodiments, the emissive agent is a near infrared (NIR) emissive species that such as a di- and tricarbocyanine dye, a croconium dye, or a thienylenephenylenevinylene species substituted with electron withdrawing substituents, where the emissive species is modified by addition of a hydrophobic substituent, the NIR dye being substantially dispersed within the polymersome membrane.

Hydrophobic moieties and means for attaching them to various chemical structures are well known to those skilled in the art. In some embodiments, the hydrophobic substitutent is a lipophilic group. Lipophilic groups include alkyl groups, fatty acids, fatty alcohols, steroids, waxes, fat-soluble vitamins, and the like. Other lipophilic substitutents include glycerides, glyceryl ethers, phospholipids, and terpenes.

Method for a Quantitative Detection of a Nucleic Acid Target

In a second aspect, the invention is a method for a quantitative detection of a nucleic acid target, the method comprising:

providing the quantitative molecular probe of the invention and a nucleic acid target;

contacting said quantitative molecular probe with said nucleic acid target;

hybridizing said quantitative molecular probe to said preselected nucleic acid target sequence; and

detecting a reporter signal from at least one of said first label moiety and second label moiety and a reference signal from said reference label moiety to obtain a fluorescence ratio,

analyzing said fluorescence ratio and said reference fluorescence signal and thereby quantitatively detecting said nucleic acid target.

In certain embodiments, the quantitative molecular probe is encapsulated in a polymersome vesicle comprising a shell comprising an amphiphilic polymer or in a liposome comprising a phospholipid shell.

In certain embodiments, the quantitative molecular probe is tethered to a solid surface.

In certain embodiments, the nucleic acid target is provided in at least one of a live cell, an isolated tissue, a mammal, or an embrio.

In certain embodiments, the fluorescence ratio is from 0 to 1,000,000, but this depends on a number or parameters or conditions, e.g., the camera, the dye, the number of MBs per QD, the excitation source, the filters, etc. In certain embodiments, the fluorescence ratio is from 0 to 5.

In a third aspect, the invention is a method for a quantitative determination of an effect of a substance on a nucleic acid target, the method comprising:

providing the quantitative molecular probe of the invention and a nucleic acid target;

contacting said quantitative molecular probe with said nucleic acid target;

hybridizing said quantitative molecular probe to said preselected nucleic acid target sequence to form a complex;

optionally contacting said nucleic acid target with said substance;

contacting a complex with said substance; and

detecting a reporter signal from at least one of said first label moiety and second label moiety and a reference signal from said reference label moiety to obtain a fluorescence ratio; and thereby quantitatively determining the effect of said substance on said nucleic acid target.

In certain embodiments, the quantitative molecular probe is encapsulated in a polymersome vesicle comprising a shell comprising an amphiphilic polymer or in a liposome comprising a phospholipid shell.

In certain embodiments, the quantitative molecular probe is tethered to a solid surface.

In certain embodiments, the nucleic acid target is provided in at least one of a live cell, an isolated tissue, a mammal, or an embrio.

In certain embodiments, the fluorescence ratio is from 0 to 1,000,000, but this depends on a number or parameters or conditions, e.g., the camera, the dye, the number of MBs per QD, the excitation source, the filters, etc. In certain embodiments, the fluorescence ratio is from 0 to 5.

In certain embodiments, the substance is a chemical substance or a physical force. Non-limiting examples of chemical substances are organic and inorganic substances, drugs, solvents, biologically active agents, etc. Non-limiting examples of physical force is an exposure to heat, sound, electromagnetic force, etc.

In certain embodiments, the substance is a drug and said method is applied to cells to identify cells which display drug resistance.

Calibration Kit

In a fourth aspect, the invention is calibration kit for detection of a signal from a quantitative molecular probe, said calibration kit comprising:

a plurality of encapsulated quantitative molecular probes comprising a quantitative molecular probes according to claim 1, wherein said quantitative molecular probe is encapsulated in at least one of a polymersome vesicle comprising a shell comprising an amphiphilic polymer or a liposome vesicle, wherein said encapsulated quantitative molecular probes have a predetermined amount of said quantitative molecular probes hybridized to a preselected nucleic acid target sequence.

The invention will be illustrated in more detail with reference to the following Examples, but it should be understood that the present invention is not deemed to be limited thereto.

### EXAMPLES

### Example 1

Synthesis of quantitative molecular probes: As a precursor to the ‘quantitative molecular probe’ (QMP), an antisense molecular beacon possessing an amino-linker within its stem was synthesized using standard phosphoramidite chemistry (FAM-GTCACCTCAGCGTAAGTGATGTCG/aminoC6T/GAC-Dabcyl) (SEQ ID NO: 15). To cross-link the aminated molecular beacons to quantum dots (Qdot 655, Quantum Dot Corp.), a 10 μM sample of the beacon was reacted with a 50-fold excess of Disuccinimidyl Suberate (DSS, Pierce) in DMSO at 40° C. for 2 hours. The activated molecular beacon was then acetone precipitated, and subsequently reacted with 6 μM Amino (PEG) quantum dots (QDs) at molar ratios of 25:1, 10:1, or 5:1 molecular beacons to QD in 50 mM Sodium Borate, pH 8.2 at 37° C. for 2 hours. QD-molecular beacon conjugates (QD-QMPs) were purified from unbound molecular beacons by gel chromatography (Superdex, Amersham). The number of molecular beacons per QD was quantified spectrophotometrically. The QMPs possessed 1 to 10 molecular beacons per quantum dot depending on the initial molar ratio of molecular beacons to QDs.

To ensure the QMB samples were pure from unbound molecular beacons, an aliquot of the QMB sample was centrifuged on a Microcon YM-50 filter (50 kDa MW cutoff, Millipore). The eluent was then incubated in the presence of complimentary target and tested for the presence of molecular beacons by performing fluorescence measurements on a SPEX FluoroMax-3 spectrofluorometer (Horiba Jobin Yvon). Specifically, the maximum fluorescence intensity of the hybridized molecular beacon in the eluent was compared to that of completely hybridized molecular beacons in the stock QMB sample. Additional examples of molecular beacons (and dual labeled oligonucleotide probes) that will be tested within the QMP design are shown in Table 1. Alternative QMP designs will be tested such as: (1) QMPs with alternative reporter dyes, reference dyes, and quenchers, (2) QMPs with the reference dye located at alternative positions (e.g. within the loop/targeting region of the QMP), or (3) “shared stem” QMPs¹¹. Chemistries utilizing longer cross-linkers between the molecular beacon and reference dye and lower ratios of molecular beacons to reference dye can be used if hybridization kinetics create a problem.

TABLE 1

Example Oligonucleotide Sequences and
Modifications

| | |
|---|---|
| Oligonucleotide | Oligonucleotide |
| Name | Sequence (5′ - 3′) |

SEQ ID NO: 15 variants

| | |
|---|---|
| LucQMP-NH2Stem +2 | Reporter^{†}-GTCACCTCAGCGTAAGTGATG |
| | TCG/aminoC6T*/GAC-Quencher^{‡} |
| LucQMP-NH2Stem −1 | Reporter^{†}-GTCACCTCAGCGTAAGTGATG |
| | TC/aminoC6T*/GTGAC-Quencher^{‡} |
| LucQMP-NH2NoStem1 | Reporter^{†}-CTCAGCGTAAGTGATGTC/ |
| | aminoC6T*/-Quencher^{‡} |
| LucQMP-NH2NoStem2 | Reporter^{†}-CTCAGCGTAAGTGATG/ |
| | aminoC6T*/C-Quencher^{‡} |
| LucQMP-biotinStem | Reporter^{†}-GTCACCTCAGCGTAAGTGATG |
| +2 | TCG/biotinT/GAC-Quencher^{‡} |
| LucQMP-biotinStem | Reporter^{†}-GTCACCTCAGCGTAAGTGATG |
| −1 | TC/biotinT/GTGAC-Quencher^{‡} |
| Control QMP | Reporter^{†}-GTCACCTCAGCGTAAGTGATG |
| | TCGTGAC-Quencher^{‡} |
| QMP Target | TGGACATCACTTACGCTGAGTA |

SEQ ID NO: 26 variants

| | |
|---|---|
| MiR20-NH₂ | Reporter^{†}-GTCACCCTGCACTATAAGCA |
| | CG/aminoC6T*/GAC-Quencher^{†} |
| MiR-20 Synthetic | TAAAGTGCTTATAGTGCAGGTAG |
| Target | |
| MiR-20 G-T Mut-Syn | TAAAGTGCTTATATTGCAGGTAG |
| Target | |
| MiR-20 A-C Mut-Syn | TAAAGTGCTTCTAGTGCAGGTAG |
| Target | |

*aminoC6T is an internal amine attached to the dT-base with a 6-carbon linker
^{†}Several reporter fluorophores will be tested, specifically, FAM, Bodipy, Cy5, Texas Red and Cy5.5.
^{‡}Several quenchers will be tested, specifically, dabcyl, Iowa Black FQ, Iowa Black RQ and BHQ-1.

Example combinations of reference dye, quencher, and reporter dye are shown in Table 2 below.

TABLE 2
Combinations of dye/quenchers to be tested

| Reporter dye | Quencher | Reference Dye |
|---|---|---|
| (Abs/Emm)^{†} | (Abs)^{†} | (Abs/Emm)^{†} |
| Cy3 (550 nm/564 nm) | Iowa Black FQ (531 nm) | QD525 (—/525 nm) |
| Cy3 (550 nm/564 nm) | Iowa Black FQ (531 nm) | QD800 (—/800 nm) |
| Cal610 (590 nm/610 nm) | Iowa Black RQ (656 nm) | QD525 (—/525 nm) |
| Cal610 (590 nm/610 nm) | Iowa Black RQ (656 nm) | QD800 (—/800 nm) |
| Cy5 (648 nm/668 nm) | Iowa Black RQ (656 nm) | QD525 (—/525 nm) |
| Cy5 (648 nm/668 nm) | Iowa Black RQ (656 nm) | QD800 (—/800 nm) |

^{†}QD (Invitrogen), Cy3 &Cy5 (Amersham), Iowa Black RQ/FQ &Cal610 (IDT)

### Example 2

Synthesis of quantitative molecular probes: As an alternative to QD-QMPs, another embodiment consists of neutravidin-based QMPs, where fluorescently-labeled neutravidin serves as the reference dye. Cy5.5-neutravidin was synthesized by first reacting 33 μM neutravidin with Cy5.5 N-hydroxysuccinimide (Amersham) at molar ratios of 3:1, 5:1, 10:1, and 20:1 in PBS, pH 7.4 for 2 hours. The Cy5.5-neutravidin conjugate was purified from free Cy5.5 by gel chromatography (PD-10, Amersham) and the labeling ratio was determined spectrophotometrically. The number of Cy5.5 dyes per neutravidin varied from 0.5 to 2. To attach the aminated-molecular beacons to the Cy5.5-neutravidin conjugate, 33 μM molecular beacons were reacted with a 10-fold excess of NHS-biotin (Pierce) in DMSO at 40° C. for 2 hours. The molecular beacon-biotin conjugate was purified by gel chromatography (NAP-5, Amersham). Alternatively, molecular beacons were synthesized with a biotin label directly incorporated into the probe. The molecular beacons and Cy5.5-neutravidin were mixed at a ratio of two molecular beacons per neutravidin and purified by gel chromatography (Superdex, Amersham). Additional tests will be performed where Neutravidin is labeled with the an amine reactive form (e.g. NHS) of various fluorophores, e.g Cy5.5, Alexa 660, FAM, TRITC, Texas Red, and Alexa 680.

### Example 3

Synthesis of quantitative molecular probes: Antisense Firefly luciferase (pGL3-Luc 235-252, promega) and antisense c-myc (564-581, GenBank Accession V00568) molecular beacons were labeled at the 5′-end with a CAL Fluor® Red 610 (Biosearch Technologies) fluorophore, Cal610, and at the 3′-end with Iowa Black RQ quencher, IAbRQ. In addition, a biotin linker was inserted within the 3′-stem. The luciferase antisense molecular beacon sequence and labeling scheme was /Cal610/GTC ACC TCA GCG TAA GTG ATG TCG/ibiodT/GA C/3IabRQ. The c-myc antisense molecular beacon sequence and labeling scheme was /Cal610/GTC ACG TGA AGC TAA CGT TGA GGG/ibiodT/GA C/3IabRQ. Luciferase and c-myc target oligonucleotides were synthesized with the sequences, GTC ACG ACA TCA CTT ACG CTG AGT TT and GTC ACC CTC AAC GTT AGC TTC ACT TT, respectively. Antisense c-myc 2′-O-methyl oligonucleotides were synthesized with the sequence GTG AAG CTA ACG TTG AGG (SEQ ID NO: 27).

Biotinylated molecular beacons were cross-linked to QD800 streptavidin conjugates (Invitrogen). Specifically, 10 μM samples of the molecular beacon were incubated with 1 μM QD800 streptavidin conjugates at molar ratios of 6:1 or 15:1 in 50 mM sodium borate, 0.05% Tween, pH 8.3 at 4° C. overnight. QD800-molecular beacon conjugates, i.e. QMPs, were then purified from unbound molecular beacons by gel chromatography (Superdex, GE Healthcare). The concentration of the purified QMP was determined by measuring the absorbance of QD800 (ε₄₀₅ₙₘ=8,000,000 cm⁻¹M⁻¹) on a Cary100 spectrophotometer (Varian).

To ensure the QMP samples were pure from unbound molecular beacons, an aliquot of the QMP sample was centrifuged on a Microcon YM-50 filter (50 kDa MW cutoff, Millipore). The eluent was then incubated in the presence of complimentary target and tested for the presence of molecular beacons by performing fluorescence measurements on a SPEX FluoroMax-3 spectrofluorometer (Horiba Jobin Yvon). Specifically, the maximum fluorescence intensity of the hybridized molecular beacon (Cal610 Exc.=590 nm, Emm.=615 nm) in the eluent was compared to that of completely hybridized molecular beacons in the stock QMB sample.

The emission profile of each QMP was acquired on the FluoroMax-3 spectrofluorometer by setting the excitation wavelength to 590 nm and recording the emission from 600 nm to 825 nm. These experiments were carried out in 50 mM sodium borate, 100 mM NaCl, 0.05% Tween, pH 8.3 using 10 nM QMP in the presence or absence of 100 nM complementary target. A representative emission spectrum for a QMP consisting of QD800 as the reference dye and a unimolecular probe labeled with Cal610 as the reporter dye and IabRQ as the quencher is shown in FIG. 4. In the absence of target the fluorescence emission of Cal610 was low, whereas in the presence of complementary target there was a significant enhancement in its fluorescent intensity. In both cases the emission of QD800 remained nearly unchanged. Despite the positioning of QD800 in the vicinity of the MB's quencher, comparison of the emission spectrum of QD800-QMBs with QD800 alone indicated less than a 7% quenching of the QD800 fluorescence in this embodiment.

### Example 4

Synthesis of quantitative molecular probes: To cross-link thiolated molecular beacons to fluorescent (reference) nanoparticles/dyes, aminated dyes/nanoparticles (e.g. Amino (PEG) quantum dots (QDs), fluorescently labeled dendrimer, etc.) are first reacted with SMCC or SATA. The activated nanoparticles/dyes are then purified and reacted with an excess of thiolated molecular beacons. Any labeling ratio can be used depending on the desired end product. Reference dye-molecular beacon conjugates (QMPs) can be purified from unbound molecular beacons by gel chromatography (Superdex, Amersham).

### Example 5

Synthesis of quantitative molecular probes: The oligonucleotides listed in Table 3 will be synthesized. The oligonucleotide sequence listed is complementary to miR-21. Initially, all of the oligonucleotides will be synthesized using standard phosphoramidite chemistry. Additional chemistries will also be evaluated as described below. The oligonucleotides Mir21-NH2Stem+2 and MiR21-NH2Stem-1 differ only in the position of the internal amine that will be used for conjugation to the reference dye/nanoparticle. The specific combination of fluorophores, quenchers, and reference dyes that will be examined are listed in Table 4. If desired, the molecular beacon specificity can be improved by varying the length and sequence of the stem and loop domains^{11,12}.

TABLE 3
Oligonucleotide Sequences and Modifications

| Oligonucleotide | Oligonucleotide |
|---|---|
| Name | Sequence (5′ - 3′) |
| SEQ ID NO: 18 | |
| MiR21-NH2Stem +2 | Reporter^{†}-GTCACCAACATCAGTCTGATAAG |
| | CG/aminoC6T*/GAC-Quencher^{†} |
| MiR21-NH2Stem −1 | Reporter^{†}-GTCACCAACATCAGTCTGATAAG |
| | C/aminoC6T*/GTGAC-Quencher^{†} |
| MiR21-NH2NoStem | Reporter^{†}-CAACATCAGTCTGATAAGC/ |
| | aminoC6T*/-Quencher^{†} |
| Control MB | Reporter^{†}-GTCACCAACATCAGTCTGATAAGC |
| | GTGAC-Quencher^{†} |
| MiR-21 Synthetic | UAGCUUAUCAGACUGATGUUGA |
| Target | |
| MiR-21 Mut-Syn | UAGCUUAUCATACUGATGUUGA |
| Target | |

*aminoC6T is an internal amine attached to the dT-base with a 6-carbon linker
^{†}The combination of reporter dyes and quenchers to be tested are listed in Table 4.

Fluorescently labeled Dendrimers: 20 μM PAMAM Dendrimers (generation 5, Sigma) will be incubated with the NHS-ester form of Cy5 and Alexa 488 at labeling ratios 2.5:1, 5:1, 10:1, and 20:1 dye-to-dendrimer in 0.1 M Sodium Phosphate, pH 8.0. Fluorescently labeled-dendrimers will be purified from free dye by gel chromatography (PD-10, Amersham) and concentrated on a Microcon (YM-30) ultrafiltration centrifugal device. The ratio of dye to dendrimer will be determined spectrophotometrically. The fluorescent properties of each dendrimer conjugate (100 nM) will be examined on a SPEX fluorimeter (Jobin Yvon). The labeling ratios that result in a maximum fluorescent signal for each dye will be used for all subsequent conjugations. Additional labeling ratios will be tested if necessary.

TABLE 4
Combinations of dye/quenchers to be tested with
corresponding flow cytometry filter sets

| | Reporter dye | | Reference |
|---|---|---|---|
| Dye | (Abs/Emm)^{†} | Quencher^{†} | (Abs/Emm)^{†} |
| PAMAM | Alexa 488 (495/519) | BHQ-1 | Cy5 (649/670) |
| QMBs | Alexa 532 (527/553) | BHQ-2 | Cy5 (649/670) |
| | Cy5 (649/670) | Iowa Black RQ | Alexa 488 (495/519) |
| QD-QMBs | Alexa 488 (495/519) | BHQ-1 | QD655 (—/655) |
| | Alexa 532 (527/553) | BHQ-2 | QD655 (—/655) |
| | Cy5 (649/670) | Iowa Black RQ | QD525 (—/525) |

^{†}Alexa and QD (Invitrogen), Cy5 (Amersham), Iowa Black RQ (IDT), BHQ (Biosearch Technologies)

Molecular Beacon-Quantum Dot/Dendrimer Conjugates: QD- and PAMAM-QMPs will be synthesized with the reporter dye, quencher, and reference dye combinations listed in Table 4. To cross-link the aminated molecular beacons to QDs (Qdot Corp), a 10 μM sample of the beacon will be reacted with a 50-fold excess of Disuccinimidyl Suberate (DSS, Pierce) in DMSO at 40° C. for 2 hours. The activated molecular beacon will then acetone precipitated, and subsequently reacted with amine (PEG)-QDs at molar ratios of 50:1, 25:1, 10:1, and 5:1 in 50 mM Sodium Borate, pH 8.2 at 37° C. for 2 hours. QD-molecular beacon conjugates will be purified from unbound molecular beacons by gel chromatography (Superdex, Amersham). The number of molecular beacons per QD will be quantified spectrophotometrically. Analogous conditions will be used to conjugate molecular beacons to PAMAM dendrimers.

To ensure that the molecular beacons remain conjugated to the reference dye/nanoparticle over extended periods of time, the QMPs will be concentrated on a microcon filter (Millipore, YM-50) after 1-week and 1-month. The eluent will be tested for the presence of molecular beacons that have been released from the reference dye/nanoparticle via fluorescence measurements.

### Example 6

Oligonucleotide Probe Design and Targeting/Decoy Strategies: Several QMP designs and targeting/decoy strategies will be explored as a means to improve their signal-to-background in living cells. In terms of QMP design, the two major variables that will be tested are the length of the targeting region (i.e. the loop) and the backbone chemistry. It is envisioned that a shorter molecular beacon (or dual-labeled oligonucleotide probe) will lead to an improved specificity and will be less likely to bind to SNPs (i.e. a lower background). Of course, shorter molecular beacons will also exhibit a lower affinity for their target and potentially not bind efficiently (i.e. lower signal). Therefore, it could prove to be quite important that the probe length be optimized and as a result we plan to explore the effect of this design parameter on the signal-to-background. We also plan to test several backbone chemistries in order to evaluate the importance of degradation in vivo. Unmodified molecular beacons (although shown to work in vivo) are susceptible to degradation and may have an undesirably high background. Therefore, we plan to explore whether or not nuclease resistant molecular beacons exhibit a reduced background. It should be noted that nuclease resistant molecular beacons (e.g. 2′-O-methyl) have previously been tested in living cells^{7, 10, 23}.

In addition to directly modifying the molecular beacon, two additional methods will also be tested in attempt to improve the signal-to-background: (1) Each mRNA transcript will be targeted with up to three molecular beacons. This measure will certainly increase the signal, presumably three-fold. This approach to improving the signal elicited by molecular beacons has recently been reported²¹. It should be noted, however, that extra precaution will be taken when attempting to quantify mRNA expression since not every transcript may be bound by all three molecular beacons (2) Decoy (nonsense) molecular beacons with only a quencher will be introduced into the cells along with the antisense molecular beacon. It is anticipated that the decoy molecular beacons will help reduce non-specific interactions that could be responsible for generating a high background signal. Further, it is hypothesized that the presence of additional quenchers could help reduce intracellular autofluorescence allowing for improved sensitivity.

### Example 7

HIV Tat-peptide-QMP Synthesis: In one QMP embodiment, the biotinylated tat-peptide (biotin-YGRKKRRQRRRC) was conjugated to the QD-QMPs by first incubating QMPs with a 10-fold excess of biotin-tat. The tat-QMP conjugates were subsequently purified on a Superdex column (Amersham).

To examine whether the tat-peptide could be used to transport QMPs into cells, the tat-QMP conjugates (50 nM) were incubated with NIH 3T3 cells and imaged at various time points (1, 2, 3, and 24 hours). Maximum intracellular fluorescence was detected after 24 hours although a faint signal could be detected as early as 1 hour. No signal was observed with QMPs that were not conjugated to the tat-peptide. The QMPs were localized predominantly in the cytoplasmic compartment with little or no nuclear localization. This is in agreement with previous reports where tat-peptides were utilized for internalization of fluorescently labeled oligonucleotides^{15, 24, 25}. Some punctate staining was observed within the cytoplasmic compartment, perhaps indicating endosomal entrapment. Endosomal disruption agents (e.g. chloroquine, sucrose, and HA2-peptide, PPAA, etc.) can be used to encourage more efficient cytoplasmic delivery. HIV tat-peptide concentrations more than 10-fold higher than we used are commonly reported for the internalization of oligonucleotides.

### Example 8

HIV Tat-peptide-QMP Synthesis: The tat-peptide sequence will be amended to possess a cysteine residue at the carboxy-terminus (tat-cys, YGRKKRRQRRRC). This will facilitate conjugation of the peptide to dendrimers, quantum dots, or other QMP embodiments. The tat-peptide will be conjugated to the QMPs by first reacting 150 nM QMPs with 1 mM SMCC for 1 hour in 0.1 M Sodium Phosphate, pH 7.2. The activated QMPs will then be purified by gel chromatography (Nap-5, Amersham) and incubated overnight with a 10-fold excess of tat-cys peptide in PBS, pH 7.2. The tat-QMP conjugates will subsequently be purified on a Superdex column (Amersham).

Tat-mediated internalization of QMPs: To determine the rate and extent of QMP internalization in a particular cell line, the cells will be incubated with the QMP-tat construct (or QMPs with no tat-peptide as a control) at 100 nM for 30-minutes, 1-hour, 2-hours, 4-hours, and 24-hours at 37° C. Internalization of QMPs will be analyzed by fluorescence microscopy and flow cytometry by using the reference dye as a marker.

To ascertain whether QMPs that are introduced into cells using tat-peptide are predominantly trapped within endosomes, cell studies will be conducted with and without endosomal disruption agents. The intracellular localization of QMPs cells will be determined by labeling the cells with FM4-64, a general marker of endocytosis, and imaging by fluorescence confocal microscopy. The amount of QMPs in the cytoplasm and endosomes will be quantified by determining the total fluorescence intensity in each compartment. Parallel studies will be conducted with lysosomotropic agents, chloroquine (0 to 400 μM), sucrose (0 to 500 mM), the Influenza HA₂ peptide (i.e. the N-terminal 20 amino acids of the influenza virus hemagglutin protein HA₂, 5 μM), and the pH-sensitive polymer PPAA^{26, 27}. It has been reported that the fusogenic peptide HA₂ and PPAA do not induce any cytotoxicity, whereas endosomal disruption agents such as chloroquine at effective doses have been shown to be cytotoxic in multiple cell lines. HA₂ peptides have also already demonstrated their utility in delivering cargo as big as nanoparticles into the cytoplasm²⁸. To evaluate the cytotoxicity of each QMP assay described, a cell viability assay will be performed (CellTiter-Glo, Promega). An optimal QMP internalization protocol will be selected based on the criteria of toxicity, total QMP internalization as determined by flow cytometry, and the extent of cytoplasmic delivery.

Alternative tat-QMP synthesis chemistries will also be tested. Specifically, SATA or SPDP (Pierce Biotechnology) will be tested as cross-linking agents in place of SMCC. Another option is to use QD-streptavidin conjugates with biotinylated molecular beacons and tat-peptides.

If the extent of QMP internalization is low (i.e. cells labeled with QMPs are not significantly different in fluorescent intensity from negative controls with no QMP) across all the cell lines, higher concentrations of QMPs will be tested (0.5 μM). Alternative ligonucleotide chemistries will also be tested including those with uncharged backbones (e.g. peptide nucleic acids or morpholinos). It is possible that the negative charge of the oligonucleotide may interfere with the positively charged cell penetrating peptide although according to our preliminary studies and other reports this is not a significant problem. If only some cell lines exhibit poor internalization of QMPs, then alternative cell-penetrating peptides (e.g. polyArg, model amphipathic peptide, and signal sequence hydrophobic region) may be tested with these cell lines. Additional options may include electroporation, transfection agents, and Streptolysin O. Each of these approaches has previously been used to introduce nanoparticles into cells²⁹⁻³¹.

### Example 9

QMP Standardization Curves: In order to accurately quantify the number of RNA targets in single cells, two standardization curves can be constructed; one curve that correlates the total reference dye fluorescence (e.g., QD800 (Invitrogen Corp., Carlsbad, Calif.) to the quantity of QMPs (i.e. QDs) within the respective region of interest, ROI, and one curve that correlates the reporter dye:QD fluorescence (e.g F_{Cal610}/F_{QD800}) to the number of nucleic acid targets hybridized per QMP. In one approach the standardization curves are established directly on the microscope by microinjecting predetermined amounts of QMPs and targets into paraffin oil and acquiring fluorescent images in the reference dye (e.g. QD800) and reporter (e.g. Cal610) channel, respectively (a representative image of a water-in-oil bubble is shown in the inset of FIG. 6A).

To construct a standardization curve for the reference dye, various concentrations of QMPs (e.g. 22 pM to 50 nM in 50 mM Sodium Borate Buffer supplemented 100 mM NaCl, 0.05% Tween, and 0.1 mg/mL BSA) were injected into paraffin oil in a Mattek glass-bottom dish using an FemtoJet and Injectman NI 2 (Eppendorf) microinjection system. Fluorescent images of the QMP water-in-oil bubble were acquired on a fluorescence microscope equipped with a digital camera. Images of reference and reporter dyes were acquired using the appropriate filter sets. A third image was also typically acquired to correct for autofluorescence in cell studies. These images were analyzed with NIH ImageJ. Specifically, a region of interest (ROI) was drawn around each bubble in the reference image and the total fluorescent intensity was measured using ImageJ. Similarly, the total fluorescence intensity from an equal sized ROI that was drawn around a “background” region was also measured. The background subtracted fluorescence measurement for each bubble, F_{QD800}, was then plotted versus the number of QD800 in the respective bubble (FIG. 6A). The number of QD800 in each bubble was determined by measuring the diameter of each bubble in IPLab, calculating the volume (assuming a spherical geometry), and then multiplying the volume by the concentration of QD800 injected.

To construct the ratiometric standardization curve, various concentrations of QMP, e.g. 25 nM and 50 nM, were hybridized to varying amounts of nucleic acid target (e.g. 24, 12, 6, 4.8, 3.6, 2.4, 1.2 and 0 target per QMB) in 50 mM Sodium Bicarbonate Buffer (supplemented with 100 mM NaCl, 0.05% Tween, and 0.1 mg/mL BSA) for 4 hours at room temperature prior to being injected into paraffin oil. Reference and reporter images are acquired and analyzed as described above. The fluorescence ratio, e.g. F_{Cal610}/F_{QD800} (FIG. 6B), is then plotted versus the number of nucleic acid targets per QMB for each of the samples tested.

### Example 10

QMP Standardization Curves: To generate standardization curves for quantifying QMP signals via fluorescence microscopy and/or flow cytometry we can synthesize, and use fluorescent calibration beads (e.g. polymersomes, liposomes) with predefined amounts of encapsulated QMP-RNA hybrids. Two standardization curves will be established prior to each application on the flow cytometer and microscope. (1) The total fluorescence intensity of the reference dyes within each polymersome (I_{reference}) will be correlated to the number of QMPs within the polymersome/cell. (2) The ratio of the reporter fluorescence intensity (Iᵣₑₚₒᵣₜₑᵣ) to I_{reference} will be correlated to the fraction of QMPs prehybridized to synthetic RNA in each polymersome. The combination of the two curves will provide information on RNA copy number.

Synthesis of calibration polymersomes: We plan to generate monodisperse unilamellar polymersomes with a known amount of encapsulated QMPs and complementary oligonucleotide target. First, giant (1-50 μm) unilamellar polymersomes will be prepared with 4-methoxy benzylolester terminated poly(butadiene-b-ethylene oxide) diblock copolymer (BdEOOCH3B; Polymer Source) or other suitable polymer by electroformation³² (or hydration). Specifically, BdEOOCH3B will be suspended in chloroform (4 mg/mL) and deposited on platinum wire electrodes by evaporating the solvent under nitrogen and vacuum drying overnight. Giant vesicles will then be formed by applying an alternating electric field to the electrodes (10 Hz, 10V). This method is preferred for our application because very few polymersomes are generated with diameters under 10 microns. Therefore, fairly monodisperse polymersomes with a 5 μm diameter can be obtained using a Mini-Extruder (Avanti) with a 5 μm Polycarbonate Membrane. Electroformation will be performed in the presence of QMPs (0, 5, 10, 50, 100, 500 nM and 1 μM) so that they will be encapsulated during vesicle formation. Separate QMP samples (100 nM QMPs) will be pre-hybridized with different amounts of complementary target (0, 25, 50, 75, 100, and 400 nM). The BdEOOCH3B polymersome membrane will be cross-linked using a K₂S₂O₈ initiator and a redox couple, Na₂S₂O₅/FeSO₄³³. This cross-linking procedure allows for the generation of extremely stable polymersomes that can withstand the high shear forces experienced in flow cytometry, which often lead to liposomal disruption.

Characterization of calibration polymersomes: Polymersome size and encapsulated QMP concentration will be validated by several methods. Specifically, particle size will be examined using a particle size analyzer (MasterSizer, Malvern) and by bright field microscopy. Bright field images of the polymersomes will be obtained on an Olympus IX81 microscope equipped with a 60× oil immersion objective. The mean size and standard deviation of the polymersome size distribution will be determined using IPLabs software.

The concentration of QMPs within the polymersomes will also be quantified using two approaches. The first approach takes advantage of the 4-hydroxy-benzoic-acid that is attached to the polymer as a means to determine its concentration. This UV chromophoric group absorbs at 255 nm with an ε=24,000 M⁻¹cm⁻¹. The concentration of polymersomes in the sample can be determined by assuming each polymersome has a 5 μm diameter and each polymer within the polymersome has a cross-sectional surface area of 1 nm² as previously reported. Since the reference dye optical properties will also be known, the QD:polymersome molar ratio and the encapsulated quantum dot concentration can easily be calculated. As an alternative method, the concentration of QMPs within polymersomes can be quantified via fluorescence microscopy. We have already established a standard curve on our microscope correlating total fluorescence intensity detected to the number of QD-QMPs (FIG. 6A). This was accomplished by microinjecting aqueous solutions of QD-QMPs with a range of concentrations into oil and taking fluorescent images with the QD filter set. The diameter of the bubble was measured and the total fluorescence within a ROI drawn around the bubble was recorded using IPLabs software. Since the QD concentration and bubble volume is known, the number of QDs per bubble was easily calculated. We can establish similar standard curves for any reference dye. Thus, measuring the total fluorescence intensity of each polymersome and correlating the value to the standardization curve can allow for the number of dyes within each polymersome to be quantified. These polymersomes can then potentially be used to establish the standardization curves for subsequent fluorescent microscopy or flow cytometry experiments.

Quantitative flow cytometry Calibration Curves: Two calibration curves will be generated prior to flow cytometry experiments, using the calibration polymersomes described above. The first curve will be generated by analyzing polymersome samples with different concentrations of encapsulated QMPs (e.g. 0, 5, 10, 50, 100, 500 nM and 1 μM). The calibration curve will correlate the mean fluorescence intensity of the polymersome (in the reference dye channel) to the number of QMPs in the polymersome. The coefficient of variation will be used to represent the precision in our approach. 10,000 events will be counted for each calibration sample.

A second calibration curve will be generated by analyzing polymersomes that contain QMPs (e.g. 10 nM) pre-hybridized to increasing amounts of target (e.g. 0, 2.5 nM, 5.0 nM, 7.5 nM, 10.0 nM, and 40.0 nM). In this case, a curve comparing the mean fluorescence intensity of the reporter dye to the reference dye (Iᵣₑₚₒᵣₜₑᵣ/I_{reference}) for each polymersome will be plotted versus the ratio of targets to molecular beacons (i.e. the fraction of hybridized molecular beacons). Again the coefficient of variation will be used to represent the precision of the measurements. We have already constructed a similar calibration curve via fluorescence microscopy (FIG. 6b) using the same water-in-oil method described above.

It is expected that these two calibration curves combined will allow us to determine the number of QMPs in each cell and the fraction of QMPs bound to RNA, or more specifically the number of RNA copies per cell.

If flow cytometry reveals a very high coefficient of variation with large overlaps between each polymersome calibration sample, then several alternatives can be explored to make more uniform and distinct polymersome samples. Specifically, if the size of the calibration polymersomes is too heterogeneous, then smaller polymersomes will be synthesized. Additional extrusion steps often result in a more uniform size distribution by removing the variations caused by small polymersomes that may be created during electroformation. If the encapsulated concentration of QMPs is uneven across polymersomes then alternative polymersome synthesis methods such as oil-in-water emulsions will be examined. Another option is to just sort the polymersomes by flow cytometry according to their fluorescence intensity and only later quantify the number of QMPs per polymersome within each fraction collected using the methods described above. With this approach, even if the polymersome size and QMP concentration are not uniform we believe the number of QMPs per polymersome will be similar. The separate fractions of polymersome-QMPs collected can subsequently be used for future calibrations on the flow cytometer or microscope.

If we are able to measure our polymersome calibration samples with small coefficient of variations, but their fluorescence intensity is too low or too high for the effective separation of each sample then additional polymersomes will be synthesized with different QMP concentrations such that the fluorescence intensities lie within the linear detection range of the flow cytometer.

### Example 11

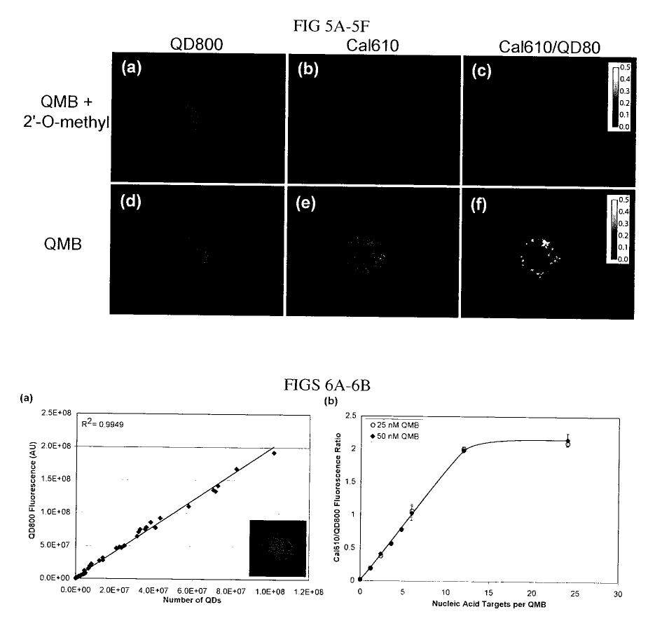

Quantitative Measurements of Endogenous RNA Expression in Living Cells via Fluorescence Microscopy: In one QMP embodiment, antisense c-myc QMPs were used to provide an absolute measure of endogenous c-myc expression in single MCF-7 breast cancer cells and to delineate the stochasticity of expression across cell populations. All measurements were made between five and ten minutes following the injection of QMBs to ensure complete hybridization. Negative control experiments were carried out by competitively inhibiting QMB hybridization with 2′-O-methyl antisense oligonucleotides targeting the same RNA sequence. While all of the cells injected with QMBs exhibited a bright fluorescent signal in the QD800 channel (representative data shown in FIGS. 5A and 5D), the cells injected with QMBs alone clearly exhibited a brighter fluorescent signal in the Cal610 channel (FIG. 5E) than the negative controls (FIG. 5B). These results are further corroborated by the quantitative analysis performed on individual cells. In cells injected with QMBs alone, calculation of RNA copy number, acquired by correlating the QD800 and F_{Cal610}/F_{QD800} measurements with externally constructed standard curves, revealed between 2,325 and 23,241 copies per cell with an average of 9,207 copies per cell. Conversely, analysis of cells co-injected with both QMBs and excess competing 2-‘O’-methyl oligonucleotides indicated that on average there were only 23±70 copies of c-myc RNA per cell and 90% of the cells analyzed had zero copies of c-myc RNA. The maximum number of copies per cell measured amongst all the negative control cells was 230 copies. An additional negative control for this study consisted of injecting luciferase antisense QMBs into MCF-7 cells. Luciferase antisense QMBs have no perfectly complementary targets that are endogenously expressed in MCF-7 cells. Therefore, as expected, quantitative analysis revealed zero copies of luciferase RNA per cell in all of the cells analyzed. Treatment of MCF-7 cells with Tamoxifen had a negative effect on c-myc expression. Although the number of c-myc RNA ranged from 0 to as many as 19,984 copies per cell, on average there was a 4.1-fold reduction in c-myc expression down to 2,223 copies per cell with 60% of the cells having zero copies. Similar fold changes, i.e. 3.9-fold, were also obtained using quantitative RT-PCR, although quantitative RT-PCR reported only 2,907±289 copies/cell for untreated MCF-7 cells and 747±101 copies/cell for cells treated with Tamoxifen. These values are 68% and 66% lower than our respective QMB measurements.

### Example 12

Quantitative Measurements of Endogenous miRNA Expression in Living Cells via Fluorescence Microscopy: QMPs will be synthesized with sequences complementary to miR-20, miR-21, miR-17-5p, miR-155, and let-7a respectively. All studies will be conducted using glass bottom culture dishes (MatTek) on an Olympus IX-81 inverted fluorescence microscope. The excitation and emission filters and dichroic mirrors will be chosen according to the reporter and reference dyes of the QMP. Following delivery of QMPs into the cells, two images will be acquired one corresponding to the reporter fluorescence (Fᵣₑₚₒᵣₜₑᵣ) and one corresponding to the fluorescence of the reference dye (F_{reference}). Ratiometric analysis on the images will then be performed. Specifically, first the background will be subtracted from each image and then an ROI will be drawn around individual cells. The background fluorescence will be determined by selecting a region of interest (ROI) in each image that does not contain any fluorescent cells. The fluorescence ratio will then be calculated by summing the fluorescence of the reporter within each ROI (Fᵣₑₚₒᵣₜₑᵣ) and dividing by the sum of the reference fluorescence within the same ROIs (F_{reference}), (Fᵣₑₚₒᵣₜₑᵣ)/(F_{reference}). The ratios calculated for multiple cells will be averaged and the standard deviation will be determined. Analogous studies will be performed with nonsense QMPs to serve as a negative control. The fluorescent values for the antisense and nonsense QMPs will be compared with the standard curves to determine the number of hybridization events predicted to occur in each cell.

To improve the detection of sensitivity of QMPs this obstacle, one option is to target polycistronic miRNA such as the miR-17-92 cluster, which has been shown to be upregulated in lung cancer. In this case, QMPs can be used to target miR-17-5p, miR-18, miR-19, miR-20, and miR92 instead one of the miRNA individually. A second option is to target sets of genes (e.g. miR-21, miR-17-5p, miR-155, miR-191, etc.), which have all been shown to be up-regulated in lung cancer.

An alternative quantification strategy, which does to require water-in-oil, calibration bead, or other externally constructed standard curves, is to correlate qRT-PCR measurements directly to QMP fluorescence to establish a new standard curve. It should be noted that even the semi-quantitative nature of the fluorescence miscroscopy measurements, without standard curves would allow QMPs to be a very effective diagnostic and biological tool.

### Example 13

Temporal Measurements of RNA Expression in Living Cells via Fluorescence Microscopy: QMPs will be introduced into MCF-7 cells. The MCF-7 cells will be treated with 10 nM estradiol following administration of QMPs. Cells will be analyzed by FACS at various time points (1, 2, 6, 24 hrs) following the administration of estradiol and the fluorescence ratio (FL-1ₘₑₐₙ/FL-3ₘₑₐₙ) will be calculated. In parallel studies, fluorescence will be monitored on a microplate reader. Maximum excitation and emission wavelengths will be selected/recorded for both the reporter dye and reference dye and the fluorescence ratio (Fᵣₑₚₒᵣₜₑᵣ−Bᵣₑₚₒᵣₜₑᵣ)/(F_{reference}−B_{reference}) will be calculated for each well at various time points following estradiol administration. The background will be determined from a well with unlabeled cells (i.e. no QMPs). Untreated cells (no estradiol) that have been transfected with QMP will serve as a negative control.

Following estradiol treatment of cells with QMPs, fluorescent microscopy images will be acquired at various time points over the course of 24 hours and a quantitative analysis will be performed. Analogous experiments will be performed on untreated cells to serve as a negative control. The number of copies of each mRNA transcript will be quantified at each time point by comparing the calculated fluorescent signals to standardization curves that will be obtained.

### Example 14

QMP-based Diagnostics (fluorescence microscopy): In one example of a diagnostic application, miRNA expression will be quantified in cells extracted from pleural effusions and transbronchial needle aspirations (TBNAs). The fluorescence microscopy approach will be particularly valuable for clinical specimens with low cellularity since miRNA can be measured at the single cell level. For samples where cellularity is not a limiting factor, then it may be possible to measure QMP-miRNA hybridization via flow cytometry. MicroRNA measurements obtained via qRT-PCR, northern blotting, and our QMP approach will be compared with cytological diagnoses.

Pleural effusions and TBNA specimens will generally be classified into three categories (benign, malignant, or indeterminate) of approximately equal sample size.

Cells from effusions and TBNAs will be obtained within 48 hours following the procedure. The cell suspensions will be washed twice with PBS and resuspended in ACL4 medium plus 5% FBS. The cells will then be divided into three aliquots for qRT-PCR, northern blotting, and QMP analysis respectively.

Cell samples allocated for qRT-PCR and northern blotting will be pelleted and miRNA will be extracted using the mirVANA miRNA isolation kit (Ambion) and analyzed as described above. Cell samples allocated for QMP analysis will be centrifuged onto glass bottom culture dishes (MatTek). Single cell measurements of miRNA with QMPs will then be conducted. The results of the three methods for measuring miRNA will be compared with cytological diagnoses in terms of sensitivity and specificity. McnNemar's test will be used to indicate the significance level for the comparison between each miRNA detection strategy and cytology.

MicroRNA measurements, particularly when using qRT-PCR and northern blot methodologies, can be marred if only a small population of cells obtained from effusions or TBNAs are epithelial cells.

### Example 15

Quantitative Measurements of Endogenous RNA Expression in Living Cells via Flow Cytometry: Flow cytometric standardization curves will be generated each day prior to the analysis of cell samples. Also, prior to flow cytometric analysis, all cell samples will be incubated with DAPI. Three-color flow cytometry will be performed using appropriate lasers and filters for the QMP reference and reporter dyes in addition to using the violet laser for DAPI (450/20 filter). Any cells that are positive for DAPI (i.e. dead cells) will be eliminated from the analysis. As with the standardization curves, 10,000 events will be counted for each cell sample. The standardization curves will be used to quantify the number of endogenous miRNA molecules in each cell.

### Example 16

QMP-based Diagnostics (flow cytometry): In one example of a diagnostic application, flow cytometry combined with QMPs will be used to quantify the level of miRNA expression in cells from pleural effusions and transbronchial needle aspirations (TBNAs). Although restricted to specimens of high cellularity, flow cytometry allows for higher throughput screening than fluorescence microscopy. MicroRNA measurements obtained via qRT-PCR, northern blotting, and our QMP approach will be compared with cytological diagnoses.

Pleural effusions and TBNAs from lung cancer patients will be obtained. Approximately, 60 samples (30 pleural effusions, 30 TBNAs) in total will be analyzed depending on availability. The specimens will generally be classified into three categories (benign, malignant, or indeterminate) of approximately equal sample size.

Cell samples allocated for qRT-PCR and northern blotting will be pelleted and miRNA will be extracted using the mirVANA miRNA isolation kit (Ambion) and analyzed as described above. The remaining cell suspension will be incubated with QMPs under conditions similar to those determined to be optimal as well as with DAPI. Flow cytometric standardization curves will be generated each day prior to the analysis of cell samples. Three-color flow cytometry will be performed using appropriate lasers and filters for the QMP reference and reporter dyes in addition to using the violet laser for DAPI (450/20 filter). Any cells that are positive for DAPI (i.e. dead cells) will be excluded from the analysis. 10,000 events will be counted for each cell sample. If any sample contains less than 10,000 cells it will be noted and potentially excluded from the study.

The F_{reference} and Fᵣₑₚₒᵣₜₑᵣ/F_{reference} will be recorded for each cell within each clinical sample and this value will be compared with the standardization curves to quantify the average number of endogenous miRNA molecules in each cell. Analogous studies will also be performed with nonsense QMPs and no QMPs to serve as a negative controls.

The results of the three methods for measuring miRNA will be compared with cytological diagnoses in terms of sensitivity and specificity. McnNemar's test will be used to indicate the significance level for the comparison between each miRNA detection strategy and cytology.

If only a small population of cells obtained from effusions or TBNAs are epithelial cells, sample enrichment may be necessary. If this is the case, the samples will be enriched using Ber-EP4-anti-human epithelial antigen (Biocare Medical). Alternatively, it may be possible to simply label the Ber-EP4-anti-human epithelial antigen with a distinct optical reporter and perform four-color flow cytometry.

While the invention has been described in detail and with reference to specific examples thereof, it will be apparent to one skilled in the art that various changes and modifications can be made therein without departing from the spirit and scope thereof.

### ABBREVIATIONS

QMP—Quantitative molecular probe
QMB—Quantitative molecular beacon
MB—Molecular beacon

### DSS—Dissuccinimidyl Suberate

DMSO—Dimethyl sulfoxide
PEG—Polyethylene glycol

### QD—Quantum dot

MW—Molecular weight
Abs—Absorbance maximum
Emm—Emission maximum
Exc—Excitation maximum

### NHS—N-hydroxysuccinimide

FAM—Fluorescein carboxamide
TRITC—Tetramethyl rhodamine isothiocyanate

### Cal610—Cal Fluor® Red 610 (Biosearch Technologies, Inc., Novato, Calif.)

### IabRQ—Iowa Black RQ Quencher™ (Integrated DNA Technologies, Coralville, Iowa)

### IabFQ—Iowa Black FQ Quencher™ (Integrated DNA Technologies, Coralville, Iowa)

ε=Extinction coefficient
NaCl—Sodium chloride
SMCC—Succinimidyl 4-N-maleimidomethyl cyclohexane-1-carboxylate
SATA—N-succinimidyl-5-acetylthioacetate
SPDP—N-succinimidyl-3-(2-pyridyldithio)-propionate
PAMAM—polyamidoamine
BHQ—Black hole quencher
SNPs—Single nucleotide polymorphisms
Tat—Transactivator of transcription
HA2—Influenza hemagglutin HA2 subunit
PPAA—poly(propylacrylic acid)
PolyArg—poly(arginine)
BSA—Bovine serum albumin
ROI—Region of interest
DNA—Deoxyribonucleic acid
RNA—Ribonucleic acid
F_{Cal610}—Total fluorescence (background subtracted) acquired within a region of interest using optical filter sets specific for the Cal Fluor® Red 610 dye
F_{QD800}—Total fluorescence (background subtracted) acquired within a region of interest using optical filter sets specific for QD800
I_{reference}—Total fluorescence (background subtracted) acquired within a region of interest using optical filter sets specific for the reference dye
Iᵣₑₚₒᵣₜₑᵣ—Total fluorescence (background subtracted) acquired within a region of interest using optical filter sets specific for the reporter dye
BdEOOCH3B—4-methoxy benzylolester terminated poly(butadiene-b-ethylene oxide) diblock copolymer
RT-PCR—Reverse transcriptase polymerase chain reaction
MiRNA—microRNA
F_{reference}—Total fluorescence (background subtracted) acquired within a region of interest using optical filter sets specific for the reference dye
Fᵣₑₚₒᵣₜₑᵣ—Total fluorescence (background subtracted) acquired within a region of interest using optical filter sets specific for the reporter dye
QRT-PCR—Quantitative reverse transcriptase polymerase chain reaction
FACS—Fluorescence activated cell sorting (BD, Franklin Lakes, N.J.)
TBNA—Transbronchial needle aspirations
DAPI—4′,6-diamidino-2-phenylindole, a fluorescent stain
AminoC6T—Amino thymidine with a 6-carbon linker spacer arm
IBiodT—Internal biotin thymidine (Integrated DNA Technologies, Coralville, Iowa)

| | | | |
|---|---|---|---|
| SEQ | | | SEQ |
| ID | | SEQUENCE | TITLE |
| NO | SEQUENCE | Name | abbreviated |

| | | | |
|---|---|---|---|
| 1 | CTG CTG GTT TTC CAC | (c-myc) | |
| 2 | GTT GAG GGG CAT CG | (c-myc) | |
| 3 | CCTACGCCACCAGCTCC | (k-ras) | |
| 4 | GCTGTATCGTCAAGGCACT | (k-ras) | |
| 5 | GAGAAAGGGCTGCCA | (Survivin) | |
| 6 | GTAGAGATGCGGTGGT | (Survivin) | |
| 7 | CTGGTGTTCCAT | (CCND1) | |
| 8 | GAAGGAGGAAGGAGG | (c-Src) | |
| 9 | CTCCATGGTGCTCAC | (Her/neu) | |
| 10 | ACACTAGCATCGTATCAG | (Her/neu) | |
| 11 | ATC AAT ATT TAA CAA | (ALF) | |
| 12 | GCA AAT ACT CAC CAT TTG | (MyoD) | |
| | G | | |
| 13 | GAGTCCTTCCACGATAC | (GAPDH) | |
| 14 | CAA AGG TTT GGA ATC TGC | (mmp-9) | |
| 15 | CTCAGCGTAAGTGATGTC | (Luc) | |
| 16 | CAGCATGAGGACCATCAG | (mam) | |
| 17 | GTACACGTCTCTGTCTGG | (TFF1) | |
| 18 | CAACATCAGTCTGATAAGC | (MiR-21) | |
| 19 | GAAACCCAGCAGACAATGTA | (MiR-221) | |
| 20 | AGCCTATGGAATTCAGTTCTCA | (MiR-146b) | |
| 21 | TCAGTTTTGCATAGATTTGCACA | (MiR-19a) | |
| 22 | AACTATACAACCTACTACCTCA | (let-7a) | |
| 23 | CCCCTATCACGATTAGCATTAA | (MiR-155) | |
| 24 | CTACCTGCACTATAAGCACTTTA | (MiR-20) | |
| 25 | ACTACCTGCACTGTAAGCACTTTG | (MiR-17-5p) | |
| 26 | CCTGCACTATAAGCAC | (MiR-20) | |
| 27 | GTGAAGCTAACGTTGAGG | (c-myc) | |
| 28 | Reporter^{†}- | | LucQMP- |
| | GTCACCTCAGCGTAAGTGATGTC | | NH2Stem +2 |
| | G/aminoC6T*/GAC- | | |
| | Quencher^{‡} | | |
| 29 | Reporter^{†}- | | LucQMP- |
| | GTCACCTCAGCGTAAGTGATGT | | NH2Stem −1 |
| | C/aminoC6T*/GTGAC- | | |
| | Quencher^{‡} | | |
| 30 | Reporter^{†}- | | LucQMP- |
| | CTCAGCGTAAGTGATGTC/amino | | NH2NoStem1 |
| | C6T*/-Quencher^{‡} | | |
| 31 | Reporter^{†}- | | LucQMP- |
| | CTCAGCGTAAGTGATG/aminoC6 | | NH2NoStem2 |
| | T*/C-Quencher^{‡} | | |
| 32 | Reporter^{†}- | | LucQMP- |
| | GTCACCTCAGCGTAAGTGATGTC | | biotinStem |
| | G/biotinT/GAC- | | +2 |
| | Quencher^{‡} | | |
| 33 | Reporter^{†}- | | LucQMP- |
| | GTCACCTCAGCGTAAGTGATGTC/ | | biotinStem |
| | biotinT/GTGAC-Quencher^{‡} | | −1 |
| 34 | Reporter^{†}- | | Control |
| | GTCACCTCAGCGTAAGTGATGTCG | | QMP |
| | TGAC-Quencher^{‡} | | |
| 35 | TGGACATCACTTACGCTGAGTA | | QMP |
| | | | Target |
| 36 | Reporter^{†}- | | MiR20- |
| | GTCACCCTGCACTATAAGCACG/ | | NH₂ |
| | aminoC6T*/GAC-Quencher^{†} | | |
| 37 | TAAAGTGCTTATAGTGCAGGTAG | | MiR-20 |
| | | | Synthetic |
| | | | Target |
| 38 | TAAAGTGCTTATATTGCAGGTAG | | MiR-20 G- |
| | | | T Mut-Syn |
| | | | Target |
| 39 | TAAAGTGCTTCTAGTGCAGGTAG | | MiR-20 A- |
| | | | C Mut-Syn |
| | | | Target |
| 40 | Reporter†- | | MiR21- |
| | GTCACCAACATCAGTCTGATAAGC | | NH2Stem +2 |
| | G/aminoC6T*/GAC- | | |
| | Quencher† | | |
| 41 | Reporter†- | | MiR21- |
| | GTCACCAACATCAGTCTGATAAG | | NH2Stem −1 |
| | C/aminoC6T*/GTGAC- | | |
| | Quencher† | | |
| 42 | Reporter†- | | Control |
| | GTCACCAACATCAGTCTGATAAGC | | MB |
| | GTGAC-Quencher† | | |

### REFERENCES

1. Neely, L. A. et al. A single-molecule method for the quantitation of microRNA gene expression. Nat Methods 3, 41-46 (2006).
2. Lu, J. et al. MicroRNA expression profiles classify human cancers. Nature 435, 834-838 (2005).
3. Tsuji, A. et al. Development of a time-resolved fluorometric method for observing hybridization in living cells using fluorescence resonance energy transfer. Biophys J 81, 501-515 (2001).
4. Tsuji, A. et al. Direct observation of specific messenger RNA in a single living cell under a fluorescence microscope. Biophys J 78, 3260-3274 (2000).
5. Sokol, D. L., Zhang, X., Lu, P. & Gewirtz, A. M. Real time detection of DNA.RNA hybridization in living cells. Proc Natl Acad Sci USA 95, 11538-11543 (1998).
6. Kehlenbach, R. H. In vitro analysis of nuclear mRNA export using molecular beacons for target detection. Nucleic Acids Res 31, e64 (2003).
7. Bratu, D. P., Cha, B. J., Mhlanga, M. M., Kramer, F. R. & Tyagi, S. Visualizing the distribution and transport of mRNAs in living cells. Proc Natl Acad Sci USA 100, 13308-13313 (2003).
8. Perlette, J. & Tan, W. Real-time monitoring of intracellular mRNA hybridization inside single living cells. Anal Chem 73, 5544-5550 (2001).
9. Matsuo, T. In situ visualization of messenger RNA for basic fibroblast growth factor in living cells. Biochim Biophys Acta 1379, 178-184 (1998).
10. Tyagi, S. & Alsmadi, O. Imaging Native [beta]-Actin mRNA in Motile Fibroblasts. Biophys J (2004).
11. Tsourkas, A., Behlke, M. A. & Bao, G. Structure-function relationships of shared-stem and conventional molecular beacons. Nucleic Acids Res 30, 4208-4215 (2002).
12. Tsourkas, A., Behlke, M. A., Rose, S. D. & Bao, G. Hybridization kinetics and thermodynamics of molecular beacons. Nucleic Acids Res 31, 1319-1330 (2003).
13. Marras, S. A., Kramer, F. R. & Tyagi, S. Efficiencies of fluorescence resonance energy transfer and contact-mediated quenching in oligonucleotide probes. Nucleic Acids Res 30, e122 (2002).
14. Molenaar, C. et al. Linear 2′ O-Methyl RNA probes for the visualization of RNA in living cells. Nucleic Acids Res 29, E89-89 (2001).
15. Nitin, N., Santangelo, P. J., Kim, G., Nie, S. & Bao, G. Peptide-linked molecular beacons for efficient delivery and rapid mRNA detection in living cells. Nucleic Acids Res 32, e58 (2004).
16. Cui, Z. Q. et al. Visualizing the dynamic behavior of poliovirus plus-strand RNA in living host cells. Nucleic Acids Res 33, 3245-3252 (2005).
17. Santangelo, P. J., Nitin, N. & Bao, G. Direct visualization of mRNA colocalization with mitochondria in living cells using molecular beacons. J Biomed Opt 10, 44025 (2005).
18. Santangelo, P., Nitin, N., Laconte, L., Woolums, A. & Bao, G. Live-cell characterization and analysis of a clinical isolate of bovine respiratory syncytial virus, using molecular beacons. J Virol 80, 682-688 (2006).
19. Santangelo, P. J., Nix, B., Tsourkas, A. & Bao, G. Dual FRET molecular beacons for mRNA detection in living cells. Nucleic Acids Res 32, e57 (2004).
20. Medley, C. D., Drake, T. J., Tomasini, J. M., Rogers, R. J. & Tan, W. Simultaneous monitoring of the expression of multiple genes inside of single breast carcinoma cells. Anal Chem 77, 4713-4718 (2005).
21. Mhlanga, M. M., Vargas, D. Y., Fung, C. W., Kramer, F. R. & Tyagi, S. tRNA-linked molecular beacons for imaging mRNAs in the cytoplasm of living cells. Nucleic Acids Res 33, 1902-1912 (2005).
22. Peng, X. H. et al. Real-time detection of gene expression in cancer cells using molecular beacon imaging: new strategies for cancer research. Cancer Res 65, 1909-1917 (2005).
23. Thomson, J. M., Parker, J., Perou, C. M. & Hammond, S. M. A custom microarray platform for analysis of microRNA gene expression. Nat Methods 1, 47-53 (2004).
24. Lewin, M. et al. Tat peptide-derivatized magnetic nanoparticles allow in vivo tracking and recovery of progenitor cells. Nat Biotechnol 18, 410-414 (2000).
25. Gao, X., Cui, Y., Levenson, R. M., Chung, L. W. & Nie, S. In vivo cancer targeting and imaging with semiconductor quantum dots. Nat Biotechnol 22, 969-976 (2004).
26. Wadia, J. S., Stan, R. V. & Dowdy, S. F. Transducible TAT-HA fusogenic peptide enhances escape of TAT-fusion proteins after lipid raft macropinocytosis. Nat Med 10, 310-315 (2004).
27. Jones, R. A. et al. Poly(2-alkylacrylic acid) polymers deliver molecules to the cytosol by pH-sensitive disruption of endosomal vesicles. Biochem J 372, 65-75 (2003).
28. Won, J. et al. A magnetic nanoprobe technology for detecting molecular interactions in live cells. Science 309, 121-125 (2005).
29. Parak, W. J., Pellegrino, T. & Plank, C. Labelling of cells with quantum dots. Nanotechnology 16, R9—R25 (2005).
30. Montet-Abou, K., Montet, X., Weissleder, R. & Josephson, L. Transfection agent induced nanoparticle cell loading. Mol Imaging 4, 165-171 (2005).
31. Agrawal, A. & Nie, S. in SPIE, Vol. 5705. (eds. A. N. Cartwright & M. Osinski) 152-1582005).
32. Discher, B. M. et al. Polymersomes: tough vesicles made from diblock copolymers. Science 284, 1143-1146 (1999).
33. Discher, B. M. et al. Cross-linked polymersome membranes: vesicles with broadly adjustable properties. J Phys Chem B 106, 2848-2854 (2002).

What is claimed is:

1. A quantitative molecular probe for detection of a nucleic acid target containing a preselected target sequence, said quantitative molecular probe being capable of assuming a closed conformation and an open conformation, said quantitative molecular probe comprising:
a) a target complement sequence of from 7 to about 140 nucleotides complementary to said preselected nucleic acid target sequence having a 5′ terminus and a 3′ terminus;
b) optionally comprising an affinity pair comprising a first affinity moiety covalently linked to said 5′ terminus and a second affinity moiety covalently linked to said 3′ terminus, said affinity pair interacting sufficiently to hold said quantitative molecular probe in the closed conformation in the absence of said nucleic acid target;
c) a label pair comprising a first label moiety conjugated to at least one of a first nucleotide of said preselected nucleic acid target sequence or said first affinity moiety and a second label moiety conjugated to at least one of a second nucleotide of said preselected nucleic acid target sequence or said second affinity moiety, provided that said first nucleotide and said second nucleotide are separated by at least 2 nucleotides and wherein said label moieties interact to affect a measurable characteristic of at least one of said label moieties such that a difference in a reporter signal is produced when said quantitative molecular probe is in the closed conformation, wherein hybridization of said target complement sequence to said target sequence causes said quantitative molecular probe to assume its open conformation, in which said label moieties do not so interact; and
d) a reference label moiety conjugated to at least one of said target complement sequence, said first affinity moiety, said second affinity moiety, said first label moiety, or said second label moiety, wherein said reference label moiety produces a reference signal which is a detectable signal qualitatively distinct from the reporter signal produced by any one of said first label moiety, said second label moiety alone or in combination with each other in the closed conformation.

2. The quantitative molecular probe according to claim 1, wherein said affinity pair is required and wherein said quantitative molecular probe is a quantitative molecular beacon and wherein said first label moiety is conjugated to said first affinity moiety and said second label moiety is conjugated to said second affinity moiety.

3. The quantitative molecular probe according to claim 1, wherein said affinity pair is not present and wherein said quantitative molecular probe is a quantitative linear oligonucleotide and wherein said first label moiety is conjugated to said first nucleotide of said preselected nucleic acid and said second label moiety is conjugated to said second nucleotide of said preselected nucleic acid target sequence.

4. The quantitative molecular probe according to claim 1, wherein said reference label moiety is a fluorophore selected from at least one of a nanoparticle, a microparticle, a quantum dot, a fluorescently labeled dendrimer, a fluorescent moiety or a fluorescently labeled moiety.

5. The quantitative molecular probe according to claim 1, wherein said first label moiety is a reporter fluorophore and said second label moiety is a quencher selected to substantially quench fluorescence of the reporter fluorophore in a closed conformation and said reference label moiety is a reference fluorophore, wherein said reference label moiety produces a signal at a wavelength sufficiently distinct from a first label moiety wavelength.

6. The quantitative molecular probe according to claim 5, wherein said reference label moiety is at least one of Cy3.5, Cy5, Cy5.5, ALEXA 660, ALEXA 680.

7. The quantitative molecular probe according to claim 1, wherein said target complement sequence is from 15 to 30 nucleotides.

8. The quantitative molecular probe according to claim 2, wherein said affinity pair comprises complementary oligonucleotide arm sequences 3 to 25 nucleotides in length.

9. The quantitative molecular probe according to claim 2, wherein said first label moiety is covalently linked to said first affinity moiety and said second label moiety is covalently linked to said second affinity moiety.

10. The quantitative molecular probe according to claim 1, wherein said reference label moiety is conjugated via covalent bonding or affinity bonding.

11. The quantitative molecular probe according to claim 1, wherein said label pair comprises either a FRET pair or a non-FRET pair.

12. The quantitative molecular probe according to claim 2, wherein said affinity pair comprises an antibody and an antigen.

13. The quantitative molecular probe according to claim 1 tethered to a solid surface.

14. The quantitative molecular probe according to claim 1, wherein said quantitative molecular probe is a unimolecular quantitative molecular probe.

15. The quantitative molecular probe according to claim 1, wherein said quantitative molecular probe is a bimolecular quantitative molecular probe consisting of a first molecule containing approximately half of said target complement sequence including said 5′ terminus, the first affinity moiety and the first label moiety; and a second molecule containing approximately half of said target complement sequence including said 3′ terminus, the second affinity moiety and the second label moiety.

16. The quantitative molecular probe according to claim 1, wherein said reference label moiety further comprises a targeting ligand.

17. The quantitative molecular probe according to claim 1, wherein said quantitative molecular probe is encapsulated in a polymersome vesicle comprising a shell comprising an amphiphilic polymer or in a liposome comprising a phospholipid shell.

18. A method for a quantitative detection of a nucleic acid target, the method comprising:
providing the quantitative molecular probe of claim 1 and a nucleic acid target;
contacting said quantitative molecular probe with said nucleic acid target;
hybridizing said quantitative molecular probe to said preselected nucleic acid target sequence; and
detecting a reporter signal from at least one of said first label moiety and second label moiety and a reference signal from said reference label moiety to obtain a fluorescence ratio,
analyzing said fluorescence ratio and said reference fluorescence signal and thereby quantitatively detecting said nucleic acid target.

19. The method of claim 18, wherein said quantitative molecular probe is encapsulated in a polymersome vesicle comprising a shell comprising an amphiphilic polymer.

20. The method of claim 18, wherein said quantitative molecular probe is tethered to a solid surface.

21. The method of claim 18, wherein said nucleic acid target is provided in at least one of a live cell, an isolated tissue, a mammal, or an embrio.

22. The method of claim 18, wherein said fluorescence ratio is from 0 to 1,000,000.

23. In a molecular probe for detection of a nucleic acid target containing a preselected target sequence having:
a) a target complement sequence of from 10 to about 140 nucleotides complementary to said preselected nucleic acid target sequence, having a 5′ terminus and a 3′ terminus;
b) an affinity pair comprising a first affinity moiety covalently linked to said 5′ terminus and a second affinity moiety covalently linked to said 3′ terminus, said affinity pair interacting sufficiently to hold said molecular probe in the closed conformation in the absence of said nucleic acid target; and
c) a label pair comprising a first label moiety conjugated to said first affinity moiety and a second label moiety conjugated to said second affinity moiety, wherein said label moieties interact to affect a measurable characteristic of at least one of said label moieties such that a difference in a reporter signal is produced when said molecular probe is in the closed conformation, wherein hybridization of said target complement sequence to said target sequence causes said molecular probe to assume its open conformation, in which said label moieties do not so interact; wherein the improvement comprises:
d) having a reference label moiety conjugated to at least one of said target complement sequence, said first affinity moiety, said second affinity moiety, said first label moiety, or said second label moiety, wherein said reference label moiety produces a reference signal which is a detectable signal qualitatively distinct from the reporter signal produced by any one of said first label moiety, said second label moiety alone or in combination with each other in the closed conformation so that said molecular probe is adapted to quantitative detect a nucleic acid target.

24. A method for a quantitative determination of an effect of a substance on a nucleic acid target, the method comprising:
providing the quantitative molecular probe of claim 1 and a nucleic acid target;
contacting said quantitative molecular probe with said nucleic acid target;
hybridizing said quantitative molecular probe to said preselected nucleic acid target sequence to form a complex;
optionally contacting said nucleic acid target with said substance;
contacting a complex with said substance; and
detecting a reporter signal from at least one of said first label moiety and second label moiety and a reference signal from said reference label moiety to obtain a fluorescence ratio; and thereby quantitatively determining the effect of said substance on said nucleic acid target.

25. The method of claim 24, wherein said quantitative molecular probe is encapsulated in a polymersome vesicle comprising a shell comprising an amphiphilic polymer or in a liposome comprising a phospholipid shell.

26. The method of claim 24, wherein said quantitative molecular probe is tethered to a solid surface.

27. The method of claim 24, wherein said nucleic acid target is provided in at least one of a live cell, an isolated tissue, a mammal, or an embrio.

28. The method of claim 24, wherein said fluorescence ratio is from 0 to 1,000,000.

29. The method of claim 24, wherein said substance is a chemical substance or a physical force.

30. The method of claim 24, wherein said substance is a drug and said method is applied to cells to identify cells which display drug resistance.

31. A calibration kit for detection of a signal from a quantitative molecular probe, said calibration kit comprising:
a plurality of encapsulated quantitative molecular probes comprising a quantitative molecular probes according to claim 1, wherein said quantitative molecular probe is encapsulated in at least one of a polymersome vesicle comprising a shell comprising an amphiphilic polymer or a liposome vesicle, wherein said encapsulated quantitative molecular probes have a predetermined amount of said quantitative molecular probes hybridized to a preselected nucleic acid target sequence.
